(19) United States
(12) Patent Application Publication
(10) Pub. No.: US 2019/0175401 A1
(43) Pub. Date: Jun. 13, 2019

(54) METHODS FOR TREATING EYE CONDITIONS

(71) Applicant: BIOLASE, INC., Irvine, CA (US)

(72) Inventors: Marcia Van Valen, Aliso Viejo, CA (US); William E. Brown, JR., Roswell, GA (US)

(21) Appl. No.: 16/272,942

(22) Filed: Feb. 11, 2019

Related U.S. Application Data
(63) Continuation of application No. 13/607,722, filed on Sep. 8, 2012, now abandoned.
(60) Provisional application No. 61/532,296, filed on Sep. 8, 2011.

(51) Int. Cl. A61F 9/007; A61N 5/06

(52) U.S. Cl. CPC A61F 9/00718; A61F 9/0079; A61N 2005/0659; A61F 9/00772; A61N 5/0613

(57) ABSTRACT

Architectures and techniques for treating conditions of the eye, such as meibomian gland disease, utilize sources of treatment energy, such as electromagnetic energy emitting devices, to implement manipulations on tissue surrounding the orbit. According to these devices and methods, the sources of treatment energy are activated to direct energy onto parts of the eye, such as the meibomian gland, to treat meibomian gland disease. The treatments can affect at least one property of the eye and allow the liquifactions to flow more freely.

### CROSS-REFERENCE TO RELATED APPLICATIONS

This application claims the benefit of U.S. Provisional Application No. 61/532,296 filed Sep. 8, 2011 (Att. Docket BI8581PR), and is related to U.S. application Ser. No. 12/540,579 filed Aug. 13, 2009 (Att. Docket BI8144P), U.S. application Ser. No. 12/204,638 filed Sep. 4, 2008 (Att. Docket BI8047P), and U.S. application Ser. No. 11/475,719 filed Jun. 26, 2006 (Att. Docket BI9936P), the entire contents all of which are hereby incorporated by reference.

This application is also related to U.S. Pat. No. 7,665,467 (Att. Docket BI9852P), U.S. Pat. No. 7,384,419 (Att. Docket B19767P), U.S. Pat. No. 7,751,895 (Att. Docket BI9846P), U.S. application Ser. No. 11/441,788 filed May 25, 2006 (Att. Docket B198781P), U.S. Pat. No. 6,389,193 (Att. Docket BI9216P), U.S. Pat. No. 6,567,582 (Att. Docket B19216CIP), U.S. application Ser. No. 12/426,940 filed Apr. 20, 2009 (Att. Docket BI8100P), U.S. Pat. No. 7,620,290 (Att. Docket B19827P), U.S. application Ser. No. 12/020,455 filed Jan. 25, 2008 (Att. Docket BI19827CIP), and U.S. Pat. No. 7,421,186 (Att. Docket B19827C1P2), the entire contents all of which are hereby incorporated by reference.

### BACKGROUND OF THE INVENTION

### 1. Field of the Invention

The present invention relates generally to medical treatments and, more particularly, to methods and apparatus for treating eye disorders such as dry eye using energies including infrared laser, ultrasound and radio-frequency, massage, and pressure.

### 2. Description of Related Art

One common ophthalmologic cause relating to dry eye disorders is known as meibomian gland dysfunction. The meibomian gland (also known as the tarsal gland) is located inside the tarsal plate at the rim of the eyelids. The meibomian gland is a sebaceous gland that is responsible for the supply of meibum. Meibum prevents the tear film from evaporating and is an oily substance. Meibum allows the eye to be closed fully, blocks the tear fluid between the edge of the eyelid and the eyeball, and prevents tears from spilling onto the cheek. There are approximately 50 glands located on the upper eyelids and 25 on the lower eyelids.

When the meibomian gland fails to perform, it causes dry eyes in humans. Dysfunction of these glands causes tears to evaporate more rapidly and leads to symptoms of dryness, burning, and irritation. There are natural bacteria that thrive on the corneal surface. These bacteria, can colonize the meibomian glands and cause problems. This failure can lead to blepharitis which is typically manifested as the infection of small pieces of the eyelid skin. When the meibomian gland swells, this leads to a condition called meibomitis which is determined by a thick, waxy secretion from the obstructed gland, Lipases references another bacterial condition that forms fatty acids which irritate the eyes and are called punctuate kerotaphy.

The number one underlying cause for dysfunction of the glands is that they get clogged up. The reason they get clogged up is usually due to hormonal changes whereby for example changes in estrogen levels can cause a thickening of the oils. It has been suggested that changes in estrogen levels also can cause a proliferation of the staphylococcal bacteria that inhabit the eyes leading to these bacteria invading the meibomian glands and thriving there. The mentioned thickening of oils plus increased populations of bacteria can gradually and undesirably decrease the secretion of desired fluids (e.g., oils) from the glands.

### SUMMARY OF THE INVENTION

Devices and methods of the present invention for treating conditions of the eye, such as Meibomian Gland Dysfunction, utilize sources of treatment energy, such as electromagnetic energy emitting devices, to implement heat and stimulation effects resulting in the release of lipids which may cause or be responsible for clogging of the meibomian gland.

The sources of treatment energy can be activated to direct energy onto parts of the eye, such as the meibomian gland, the meibomian glands located specifically on the lower lid, the meibomian glands located specifically on the upper lid, and/or the surrounding soft tissue of the orbit.

The source of treatment energy can comprise a source of electromagnetic energy, such as but not limited to a non-coherent source and/or a laser. In certain implementations the laser can be or comprise an Erbium-based pulsed laser which emits optical energy into the meibomian glands of the eye or eyelid and/or a diode-based continuous or pulsed laser which emits optical energy into the meibomian glands of the eye or eyelids. Introduction of the treatment energy into the meibomian glands can, for instance, increase or facilitate an increase in heat of the surrounding orbital tissue, thereby mitigating the effects of meibomian gland syndrome.

While the apparatus and method has or will be described for the sake of grammatical fluidity with functional explanations, it is to be expressly understood that the claims, unless indicated otherwise, are not to be construed as necessarily limited in any way by the construction of “means” or “steps” limitations, but are to be accorded the full scope of the meaning and equivalents of the definition provided by the claims under the judicial doctrine of equivalents.

Any feature or combination of features described herein are included within the scope of the present invention provided that the features included in any such combination are not mutually inconsistent as will be apparent from the context, this specification, and the knowledge of one skilled in the art. in addition, any feature or combination of features described or referenced may be specifically included, replicated and/or excluded, in any combination, in/from any embodiment of the present invention, For purposes of summarizing the present invention, certain aspects, advantages and novel features of the present invention are described. Of course, it is to be understood that not necessarily all such aspects, advantages or features will be embodied in any particular implementation of the present invention, Additional advantages and aspects of the present invention are apparent in the following detailed description and claims that follow.

### BRIEF DESCRIPTION OF THE FIGURES

FIGS. 1-4 are schematic illustrations corresponding to types of procedures that can be implemented to treat an eye according to first aspects of the present invention;

FIGS. 5-14 are schematic illustrations corresponding to types of procedures that can be implemented to treat an eye according to second aspects of the present invention;

FIG. 15 is a structural diagram showing a device which can be used to treat an eye according to certain aspects of the present invention;

FIGS. 16-18 are schematic illustrations corresponding to types of procedures that can be implemented to treat an eye according to third aspects of the present invention;

FIGS. 19 and 20 are schematic illustrations corresponding to types of structures and corresponding processes that can be implemented to treat an eye according to fourth aspects of the present invention; and

FIGS. 21-23 are schematic illustrations corresponding to types of devices and methods that can be implemented to treat an eye according to fifth aspects of the present invention.

### DETAILED DESCRIPTION OF THE INVENTION

Reference will now be made in detail to the presently preferred embodiments of the invention. Embodiments of the invention are now described and illustrated in the accompanying drawings, instances of which are to be interpreted to be to scale in some implementations while in other implementations, for each instance, not. In certain aspects, use alike or the same reference designators in the drawings and description refers to the same, similar or analogous components and/or elements, while according to other implementations the same use should not. According to certain implementations, uses of directional terms, such as, top, bottom, left, right, up, down, over, above, below, beneath, rear, and front, are to be construed literally, while in other implementations the same use should not.

As used herein, “blepharitis” refers to the inflammation that affects the eyelids. Blepharitis usually involves the part of the eyelid where the eyelashes grow. Blepharitis occurs when tiny oil glands located near the base of the eyelashes malfunction.

As used herein, “chalazion” is also known as meibomian gland lipogranuloma, and is a cyst in the eyelid that is caused by inflammation of a blocked meibomian gland, usually on the upper eyelid.

As used herein, “estrogen” refers to the steroid hormones that readily diffuse across the cell membrane.

As used herein, “eyelid” refers to a thin covering of skin that protects the eye.

As used herein, “lipases” refers to the enzyme capable of degrading lipid molecules.

As used herein, “liquefaction” refers to unconsolidated sediments that are transformed into a substance that has a reduced viscosity and/or acts like or is more characteristic of a liquid.

As used herein, “meibomia” refers to a special kind of sebaceous gland at the rim of the eyelids inside the tarsal plate, responsible for the supply of meibum.

As used herein, “meibum” refers to an oily substance that prevents evaporation of the eye's tear film.

As used herein, “sebaceous glands” refers to the glands located in the eyelids that secrete a special type of sebum into tears.

As used herein, “surrounding tissues of the orbit” refers to the tissues that hold the eye in place between muscles and the skin.

As used herein, “tarsal gland” refers to another name for meibomian gland.

As used herein, “tarsal plate” refers to two comparatively thick, elongated plates of dense connective tissue about 2.5 cm in length; one is found in each eyelid.

As used herein, “treatments” refers to any treatment that alters the tissue of the eye.

Regarding treatment of meibomian gland dysfunction via focused treatments for example, the eyelids, and/or parts of the eyelids such as the meibomian gland ducts, may be treated (e.g., lased and/or otherwise irradiated) with treatments (e.g., micro-doses of energy), taking care to attenuate or avoid any undesired distortion of functional optical) characteristics of the tissue surrounding the orbit in the process. In an exemplary implementation, sizes, arrangements, depths, and/or other characteristics of treatments (e.g., micro-doses of energy) can be adjusted so as, for example, to increase meibomia flow (e.g., liquefaction) of the meibomian glands. Following treatment, the eye may be better able to have and function with the correct fluids including lipids. For instance, according to certain implementations, relatively small spots (e.g., micro-doses, perforations, or energy-contacted areas) ranging from about 1 micron to about 1000 microns may be created with, for example, a micro-drill, laser, or needle. In other instances, alternative or additional treatments (e.g., micro-doses of energy having spot shapes) may be either similarly formed in the tissue surrounding the orbit or formed using means (e.g., a differently configured or different type of energy-source) apart, distinct or functionally distinguishable from that used to form the mentioned treatments, in the same or different locations, at the same or other points in time, and/or with the same or different sizes.

In modified embodiments, any of the treatments may have sizes (e.g., maximum diameters) the same as or smaller than about 1 micron and/or larger than about 5 microns (e.g., ranging up to about 50 microns, or up to about 400 microns, or more, in certain implementations). Laser characteristics can be adjusted according, for instance, to a depth and diameter of desired doses (e.g., cuts). For example, doses of energy formed with depths of a few microns may be generated with relatively high power densities and/or may have relatively small diameters.

Micro-doses of energy may be formed in the tissue surrounding the orbit, for example, directing relatively unfocused treatment energy through the eyelid or meibomian gland with a focal point of the treatment energy being targeted on the tissue surrounding the orbit, or they may be generated endoscopically, According to certain implementations, the focal point can be moved (e.g., advanced distally in a direction toward the tissue surrounding the orbit) as the depth of the dose (e.g., cut) increases into the tissue surrounding the orbit, in which case conically-shaped doses of energy may result, as just one example, which exemplary formations may be beneficial in certain cases. In modified embodiments, micro-doses of energy may be formed in the tissue surrounding the orbit endoscopically. Endoscopic access may be achieved through, for example, the ocular tissue surrounding the orbit. Entry also can be accomplished, for example, adjacent to or about 1 mm from the meibomian gland.

In certain implementations, micro-doses of energy may be formed in the tissue surrounding the orbit adjunctive to, for example, a meibomian gland procedure, which may involve, for example, formation of treatments in the tissue surrounding the orbit as described herein. The treatments (e.g., micro-doses of energy in the tissue surrounding the orbit) also may be adjusted, in accordance with another aspect of the present invention, to affect at least one property of the tissue of the treatment. Removal of obstructing and/or non-eye material, such as material comprising meibum lipids and/or lipid deposits, from (and/or including) the tissue surrounding the orbit may, for example, augment a liquefaction and accordingly enhance fluidics of the eye.

Low-level laser or light dosing (e.g., therapy or biostimulation) of one or more parts of the eye (e.g., the meibomian gland), further, may be performed (e.g., to map, mark, identify, anesthesize, heat, alter or rejuvenate tissues or proximate materials thereof. In a case of the tissue surrounding the orbit, a supply or flow of sebaceous liquid, for example, of the meibomian gland may be increased to thereby enhance the stimulation, health or function of the meibum. In such instances, the meibomian glands and/or their contents can be considered a target chromoform (i.e., target color, composition or type). Generally, an aspect of the invention can comprise aligning a wavelength of applied light energy with a tissue type or content (e.g., meibum) of the meibomian gland.

A type of low-level laser or light (e.g., of therapy or photo dynamic therapy (PDT) type) may be used, as an example or as another example, on or in a vicinity of (e.g., on tissue adjacent to) the meibomian gland to rejuvenate liquefaction and thereby facilitate, for example, a clear tear formation in the eye. Laser or light may be used for instance on or in a vicinity of the meibomian gland to cause or allow the meibum to soften, be dissolved, or have a consistency thereof changed so that it can or can easier be excreted (e.g., as with, by analogy, butter), thereby to rejuvenate (e.g., enhance) the liquefaction.

Light wavelengths of, for example, 670, 795, 819 and 980 nm may be employed in typical embodiments. According to one feature of the present invention, light having wavelengths within a range of about 1700 to 2000 microns (e.g., about 1710 microns, such as emitted from a diode laser) may be directed (e.g., focused) on or into the meibomian gland or its contents (e.g., to melt, dissolve, reduce a viscosity of, and/or promote flow of the contents within the meibomian gland).

The directing according to one feature is performed in a way so as not to undesirably damage other (e.g., one or more other) tissues or cell structures. According to certain embodiments the directing may be performed as a first process of a procedure, followed by a second process of the procedure being performed on or in a vicinity of (e.g., on tissue adjacent to) the meibomian gland using light having one or more characteristics different from that of the first process. For instance, the second process may be performed using a YSGG laser (e.g., an Er,Cr:YSGG having a wavelength of about 2.78 microns).

The spot size can vary, such as for instance from about 200 microns (e.g. via a handpiece with a tip) to about 30 mm (e.g., via a non-contact mode or a deep tissue handpiece). A pulsing characteristic of the light can comprise, for example, pulsing that is one or more of continuous wave or within a range of about 1-100 Hz.

Implementation of the first process, in which for instance the meibum is heated, can comprise, for example, continuous-wave or relatively low repetition-rate light. Implementation of the second process, in which for instance tissue of or adjacent to the meibum is ablated/removed, can comprise, for example, a pulse with a higher repetition rate than that of the first process. Regardless, the power typically is held below about 10 Watts (e.g., for either or both of the mentioned diode and YSGG exemplary implementations). The described light can be used, for example, to remove the outer layer of the meibomian gland and/or its content (e.g., meibum) and/or to kill bacteria that causes the gland to be clogged.

Various light sources, including for example low-level lasers and/or light-emitting diodes (LEDs), may be used separately and/or together in space and/or in time. Continuous-wave (CW) energy or pulsed energy having a relatively high peak energy may be useful in the meibomian. gland treatments. The meibomian gland may be stimulated in some cases with, for example, CW energy gated, for example, on for about 200 ms and off for about 200 ms. The stimulation may restore the meibomian gland production of meibomia to a relatively more youthful stage. The above low-level applications may also be applied to orbital surrounding tissues according to modified embodiments, such as, for example, low-level laser therapy being applied to the eyelid for meibomian gland stimulation. The technology disclosed in the above-referenced U.S. Pat. No. 7,751,895 (Att. Docket B19846P) may be used in connection with any of the low level energy applications set forth, compatible or suggested herein.

Scanning can be performed with for example a relatively small spot size. A joystick may be provided to facilitate any of the scanning implementations described herein. In other instances, a treatment or beam (e.g., of a larger spot size) can be used without scanning. Low-level light therapy may be beneficially applied to treatment of a larger portion (e.g., a relatively large or entire area) of the orbital surrounding tissue. Treatment power densities may be relatively low, being similar, for example, to power densities used in treatments of, e.g., tennis elbow, temporomandibular joint (TMJ), or tendonitis, and in representative embodiments having characteristics less than the following: a power density at the surface of the tissue being treated of about 1.47 W/cm², a power density within the tissue of about 0.39 W/cm², a dose of energy of about 23.6 J/cm² (for a 60 second laser exposure), and/or an energy of about 9 J within and about 33.5 J at the surface of the tissue being treated.

Energy can be directed to the meibomian gland during a low-impartation process for accomplishing heating to a temperature for instance of about 100 F (e.g., whereby the temperature is raised no more than about 12 F), to soften contents within the meibomian gland. The eyelid can be held back/down by the hand of an assistant, for example. A backing to protect parts of the eye (e.g., behind the target) can comprise an eye cup, a paddle and/or part of a finger. Implementations may comprise a diode laser of 810 to 980 nm wavelength at 1 to 3 W, whereby for instance the meibomian gland and/or its contents may be darkened (e.g., with a color or dye) to have an enhanced wavelength absorption characteristic, with regard to the energy, using for example a swab applicator. Energy may be imparted, for example, using a Biolase® Contoured Handpiece sized for example to match or resemble dimension(s) of the patient's eyelid. The output tip can be placed onto or up to about 10 mm away from the target during application of energy.

Apart from and/or following the above low-impartation process (which may result, for example, in a certain percentage, for example but not limited, 1 to 70, or 5 to 30, of obstructive material being removed and/or loosened (e.g., whereby a viscosity is lowered), a more focused and/or high-powered energy, such as for instance from a laser having a wavelength of about 3 micron and power of 0.1 to 1 W, may be applied to obstructing materials and/or tissues of the gland. In one example, a 100 to 600 um (e.g., 200 um) diameter tip can be used. The tip or use thereof may comprise any part (e.g., a side firing tip) of that disclosed in any one of U.S. application Ser. No. 12/426,940 filed Apr. 20, 2009 (Att. Docket BI8100P), U.S. Pat. No. 7,620,290 (Att. Docket BI9827P), U.S. application Ser. No. 12/020,455 filed Jan. 25, 2008 (Att. Docket. BI19827CIP), and/or U.S. Pat. No. No. 7,421,186 (Att. Docket B19827CIP2). Impartations from the more focused and/or high-powered energy can comprise touch-tip and/or slightly-spaced (e.g., 2-5 mm away from target) technologies and techniques.

Typically, the obstructed gland will have a white center resembling, for example, the appearance of a “whitehead” form of acne. Referenced as an acne-vulgaris lesion, the analogous “whitehead” condition can be characterized by a pore being blocked (e.g., partially obstructed or completely blocked) with trapped sebum (oil), bacteria, and/or dead skin cells, causing a tiny white spot to appear on the surface. Application of the more focused and/or high-powered energy can be for a duration of a half second or a second; then, the process may be repeated following, prior to, or during application of pressure using any known means and/or agitation and/or vibration; followed by repeating of any of the preceding in any combination and number of times/iterations until, for example, the “whitehead” condition is viewed/discerned to be corrected to an acceptable degree as observed by a user and/or equipment of a change in appearance and a change of rate of an excretion/expelling of fluid.

In one implementation, a type of low-level laser or light therapy or photo dynamic therapy (PDT) may be used to increase an efficacy of or dissolve the lipids. Entry may be through a meibomian gland or orbital surrounding area using an endoscopic laser. An anterior insertion or posterior site can be lased to cause a more direct effect on the meibomian One procedure in accordance with the present invention may comprise lasing the meibomian gland (e.g., a portion of the surrounding orbital tissue that produced meibum) in order to make clear tears be produced with the appropriate amount of liquefaction. According to one embodiment, the meibomian glands can be stained, making them a target chromoform, thereby resulting in selective treatment of the meibomian glands when exposed to optical energy,

According to a broad aspect of the present invention, one or more of the treatments may be implemented as described herein using various forms of treatment energy, such as one or more of electromagnetic radiation (e.g., ablating optical energy, thermal optical energy, low level therapeutic optical energy, or radio frequency energy), ultrasound, and magnetism, alone or in combination with acupuncture or other therapeutic interventions. Low-level therapeutic optical energy applications are described in U.S. Provisional Application No. 60/687,256 filed Jun. 3, 2005 and entitled TISSUE TREATMENT DEVICE AND METHOD (Att. Docket BI9846PR), the entire contents of which are expressly incorporated herein by reference. Embodiments may employ, as examples, laser acupuncture, light acupuncture, laser/RF acupuncture, and the like, separately and/or together in space and/or in time. In modified embodiments, any one or more of the treatments described herein may be formed with a cutting or piercing tool, such as a needle or scalpel, alone or in combination with (e.g., in space and/or time) any of the other mentioned treatment generating implements. Typically, acupuncture may be performed once a meridian or trigger point is identified. Magnets and/or magnetism, applied (e.g., separately and/or together in space and/or in time) in conjunction with the herein discussed techniques and/or ultrasound, may be beneficial as well. In particular, tissue rejuvenation may employ ultrasound, RF, laser, light, and/or magnets applied individually and/or in combination in space and/or time. Ultrasound applied to the eye, e.g., by varying a frequency of the ultrasound applied to eye tissue, may serve to recondition the eye.

According to another broad aspect of the present invention, treatments can be introduced. into the meibomian gland or orbital surrounding tissue. in exemplary implementations, each of the treatments (e.g., doses) comprises a shape, which may resemble a dot, spot, a short dash, or other object. That is, the shape may in certain embodiments not take a form of an elongated arc or a spot. For instance, a maximum length dimension of a treatment can range from about 0.01 mm to about 10 cm, a maximum width dimension can range from about 0.01 mm to about 10 cm, and a maximum depth dimension can range from about 0.01 mm up to about 10 cm (or, alternatively, up to about 115 cm). The shapes and locations may be dependent on the “mapping” of the orbital surrounding tissue wherein, for example, there are dense locations depicted by the meibomian glands or meibomian gland surrounding tissues, The eye muscles and critical eye structures may also play a role in determining shapes and/or locations of the treatments that may be required, The thermal properties of the energy injected into the tissue may require protection to eye muscles and critical eye structures.

In certain embodiments, treatments may be formed to have maximum diameters of about 1 micron to about 10 cm, and in particular implementations having maximum diameters of about 20 microns to about 20 cm. In other implementations, which may or may not consist of or comprise the application of ablating optical energy to the meibomian gland, other definitions or meanings for the term “treatments” may apply.

One or more of the treatments may be implemented using various forms of treatment energy, such as one or more of electromagnetic radiation (e.g., ablating optical energy, thermal optical energy, low level (e.g., therapeutic optical) energy, or radio frequency energy), ultrasound, and magnetic implementations.

Regarding formation of treatments using treatment energies, typical systems for providing treatment energies may comprise one or more of an electromagnetic source such as a laser (e.g., a diode laser) having a predetermined wavelength, an ultrasound device with a predetermined pulse, a heat emitting device with a pre-determined setting that interacts with desired parts (e.g., and/or lipids or materials) of the eye to form treatments, a radiofrequency module, an ultrasonic component, and combinations thereof. Electromagnetic energy sources or devices may comprise, for example, lasers having all wavelengths, such as lasers having wavelengths ranging, for example, from about 0.15 microns to about 3.2 microns. Exemplary beam (e.g., laser beam) spot sizes can range from about 0.001 mm up to about 10 cm (or, alternatively, up to about 20 cm), and exemplary energy per pulse values can range from about 0.1 mJ to about 50 mJ depending on, for example, the pulse duration and the beam (e.g., laser beam) spot size. Typical pulse (e.g., laser pulse) widths may range from about 100 nanoseconds to about 1000 microseconds. Another laser that can be utilized is the diode laser with a wavelength from 810 nm to 980 nm and energy from 0.1 watt to 10 watts in either continuous or pulsed mode. Energy may be applied, for instance, to the glands of a lower eyelid using the technology disclosed in the above-referenced U.S. Pat.. No. 7,384,419 (Att. Docket BI9767P) for low-level energy applications and/or the above-referenced U.S. Pat. No. 7,384,419 (Att. Docket BI9767P) whereby for instance a curvature of the handpiece output tip can be matched (e.g., aligned) with a curvature of the lower eyelid series of glands to be treated (e.g., with the tarsal plate at the rim of the eyelid).

Particular implementations of lasers for use on, for example, the meibomian gland may comprise Er:YAG, Er:YSGG, Er, Cr:YSGG, or CTE:YAG lasers operated at exemplary wavelengths ranging from about 2.69 microns to about 2.8 microns, and about 2.94 microns; XeCl excimer lasers operated at an exemplary wavelength of about 308 nm; frequency-shifted solid state lasers operated at exemplary wavelengths of about 0.15 microns to about 3.2 microns; excimer lasers of ArF operated at an exemplary wavelength of about 93 nm; harmonic generations of Nd:YAG or Nd:YAL or Ti:sapphire lasers operated at exemplary wavelengths of about 190 nm to about 220 nm; CO lasers operated at a wavelength of, for example, about 6.0 microns and carbon dioxide lasers operated at a wavelength of, for example, about 10.6 microns; diode lasers operated at exemplary wavelengths of about 0.8 microns to about 2.1 microns; gas lasers operated at exemplary wavelengths of about 2.6 microns to about 3.2 microns; and other gas or solid state lasers including flash-lamp and diode-laser pumped lasers operated at exemplary wavelengths of about 0.5 microns to about 10.6 microns; and optical parametric oscillation (OPO) lasers operated at exemplary wavelengths of about 2.6 microns to about 3.2 microns.

According to exemplary implementations of applying energy (e.g., optical energy) to tissues (e.g., the tissue surrounding the orbit or meibomian gland and/or material immediately-adjacent thereto), any of the phrases “plurality of treatments,” “treatments,” “tissue treatments” or “markings” can in certain embodiments refer to treatment groupings and/or treatment markings corresponding to treatment groupings. Any of these phrases can, in the same exemplary implementations and embodiments or in others, refer to two or more treatments arranged in a non-linear and non-arcuate grouping (e.g., pattern) on the tissue, and/or arranged in a plurality of non-linear and non-arcuate groupings (e.g., patterns) on and/or adjacent to the tissue. Treatments or groupings of treatments may comprise random or sundry predefined spot shapes, (straight, curved, or otherwise), or may comprise spot shapes (straight, curved, or otherwise) formed in a pattern that is pre-determined based on a treatment customized to an area.

In other implementations, which may or may not consist of or comprise the application of ablating optical energy to the meibomian gland, other definitions or meanings may apply. Typical embodiments can comprise grid-like groupings of treatments, wherein for example the individual treatments can be arranged in rows and columns in a staggered or non-staggered fashion. Other typical embodiments can comprise grid-like groupings, and/or other uniform or substantially uniform groupings, of treatments. Still further embodiments can comprise non-uniform groupings of treatments. The groupings may be formed manually and/or with the aid of automated devices such as computer controlled or aided scanners known to those skilled in the art.

Regarding formation by manual means, an output, such as, for example, a fiber optic tip in cases where the treatment is electromagnetic energy, may be used to focus electromagnetic (e.g., optical) energy onto for example the meibomian gland and/or tissue surrounding the orbit in order to form. treatments to depths of, for example, about 1% to about 99% of the meibomian gland. An exemplary implementation can comprise an Er, Cr:YSGG laser with a 200 micron quartz or sapphire (contact) tip operated at 1.25 W and 2.78 microns, wherein for example incisions may expand up to 2 mm width after laser energy is imparted with exemplary lengths of incision being about 4 mm. In other embodiments, a surgical scalpel (e.g., diamond blade) may be used to form treatments having depths as previously discussed in connection with fiber optic tip embodiments. In further embodiments, plasma technology can be used.

Regarding formation by automated scanning, typical optical systems for providing treatment energies may comprise ablative lasers having predetermined wavelengths and being focused by, for example, a tissue surrounding the orbit which is directed, for example, onto a scanner for patterning (e.g., using a mirror) onto the patient's eye. The scanner may comprise motorized mirrors and/or a refractive optical means such that laser energy is delivered (e.g., scanned) to the eye in predetermined patterns. The scanner thus can automatically direct laser energy over, for example, the meibomian gland or the tissue surrounding the orbit of the eye to generate predetermined patterns and thereby form treatments to depths of, for example, about 1% to about 99% of the meibomian gland. Operating parameters for the laser can be 0.01 watts to 10.0 watts with a repetition rate of 0 to 100 Hz. Cautery device parameters can be technique specific, and can depend upon the use and desired application. Furthermore, the output can vary depending upon the manufacturer of the cautery device.

One or more of various advantages may be realized through implementations of scanners in the context of many of the presently described embodiments, such advantages including precision, repeatability, predictability of results, uniformity of treatment sizes and/or shapes, uniformity of spacings between and/or relative positions of treatments, and speed. Moreover, scanners may be implemented to determine surface topographies and thicknesses of various layers of the eye, as known to those skilled in the art. In addition, embodiments implementing scanners may further provide a benefit of modifiability of treatments to a given patient. For instance a grouping or groupings may be formed during only a single procedure on the patient's eye (e.g., one surgical procedure during one patient visit) and, subsequently, should a need be presented, one or more follow-up procedures (e.g., implemented over multiple patient visits) may be performed on the patient's eye. These procedures may be performed in any order and/or any sequence of sub groupings, may be implemented.

Precision and efficacy of treatments may be enhanced when the depth or depths of the tissue(s) being affected (e.g., depth into meibomian gland) is/are accurately determined and controlled. In the contexts of manual generation of treatments, a surgeon may observe a color change of, for example, the tissue surrounding the orbit being treated to determine when the treatment depth reaches a desired level. In the context of procedures on the tissue surrounding the orbit, the surgeon may, for example, cease the forming or cutting of a treatment when a color change to dark (which may be more pronounced in the context of optical ablating rather than scalpel cutting) begins to change at the bottom of the treatment being formed. A darkening of hue (e.g., to a dark brown) as tissue is affected (e.g., removed) at the bottom of the treatment. may indicate, for example, less remaining meibum and a greater exposure of the underlying layer (e.g., the vascularized tissue surrounding the orbit), at which time the surgeon may decide to slow or stop altogether formation of that treatment or to stop formation altogether.

When scanners or other automated or semi-automated systems are used in connection with generation of treatments, the patient's meibomian gland thickness can be measured, for example, pre-operatively and the treatment depth controlled accordingly. In representative implementations, a scanning laser, or any other known tissue layer thickness measuring device, can be used to determine and subsequently control this depth. For example, the scanning laser may work with another optical or ultrasound device to detect the depth. Magnetic devices also may be used to the same purpose. As another alternative, a sensor may determine depth by automatically detecting, for example, a change in hue while Generally, a device such as, e.g., an optical detector, a colorimeter, an ultrasound probe, a device for generating and detecting electric and magnetic fields, and a tonometer can be used to measure depth of cut. Other methods of depth estimating include monitoring a bottom of a kerf or other topography while looking for bulging. Temperature changes also may provide an indication of depth, with a drastic change in temperature being an indication that an endpoint of the incision or kerf has been reached.

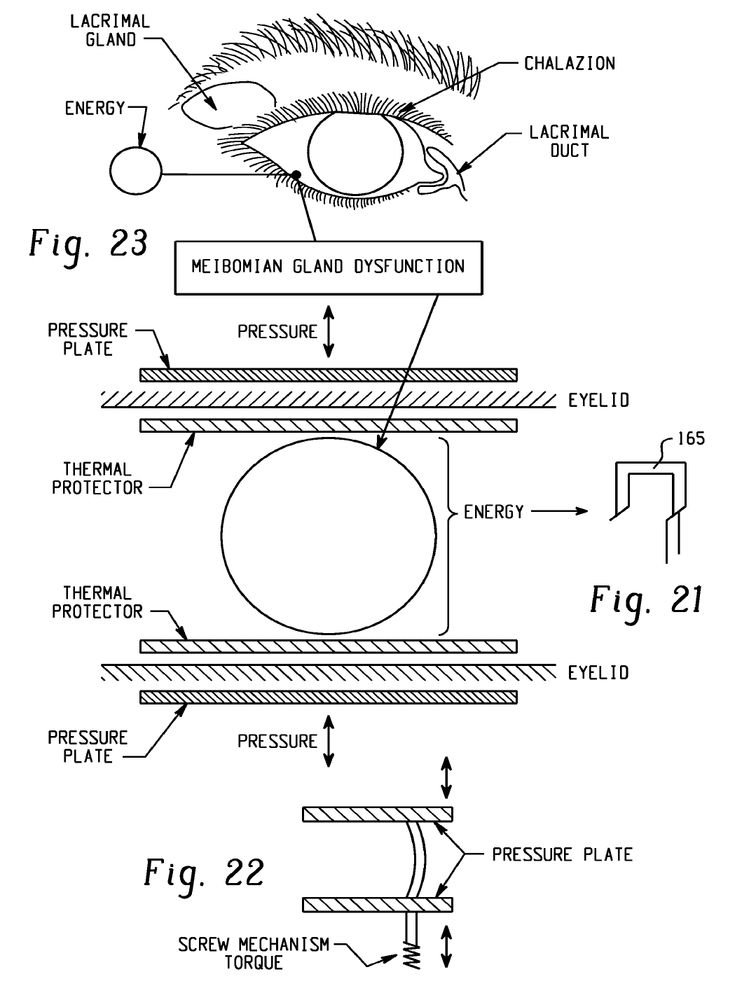

With reference to FIGS. 21, 22 and 23, according to certain examples, a camera 160, such as, for example, an intraocular fiber optic camera, may be incorporated. The camera 160 may be used, for example, to provide optical aid in conjunction with the operating site and/or to provide, for example, a determination of the incision depth in relation to the tissue surrounding the orbit. A change of color in the ocular structure, for example, can facilitate a determination of when the incisional appropriate penetration level has been reached. In other embodiments, the camera 160 intraocular or extraocular) may be configured to facilitate viewing of treatment formations, real-time or post-procedure, or to facilitate automated or semi-automated control of, for example, a procedure for forming treatments. A real-time viewing example may comprise, for example, use of an intraocular camera to facilitate real-time sub-meibomian gland visualization during formation of treatments (e.g., via laser ablation) in the meibomian gland. While monitoring the formation of a treatment using a camera, a change in color may be automatically detected and/or visually detected by a user.

In exemplary embodiments, the camera 160 may be secured, for example, to an output tip of a system (e.g., a laser system), which provides treatment energy, such as shown in FIGS. 21, 22 and 23, through a fiber optic tip 165. In FIG. 21, the output tip can comprise barbs 163 for facilitating insertion of the output tip through the tissue surrounding the orbit with relative ease but resisting removal of the barbed output tip from within the meibomian gland once inserted. Technology disclosed in the above-referenced U.S. Pat. No. 6,389,193 (Att. Docket BI9216P) and/or U.S. application Ser. No. 12/426,940 filed Apr. 20, 2009 (Att. Docket BI8100P) may be used for operation within the meibomian gland.

The fiber optic camera 160 can be integrated into the handpiece such as depicted at A1 or can branch from the output tip such as shown at B1. Similar constructions can be implemented into an oval shaped output tip, as depicted in FIG. 22. Other similar constructions can comprise a fiber optic camera or fiber optic camera lens 160 surrounding the fiber optic tip 165. According to any of the embodiments described herein, the camera 160 may comprise a visualization fiber optic leading to a remotely disposed (e.g., not on the output tip) camera. The fiber optic may be disposed in a cannula, which further may contain one or more of a treatment-energy waveguide (e.g., a fiber optic tip), a visualization light source, a fluid output and an aspiration source a calibrated aspiration source). Fluids, such as liquids (e.g., water) and/or air, can be directed over a lens of the intraocular camera and/or across a field of view of the intraocular camera to create a better viewing area and/or aspiration can be applied for removing fluids from a vicinity of the lens or field of view. In addition to or as an alternative to the discussed fluid and aspiration structures and techniques for use in combination with, for example, an intraocular camera lens, water repelling coatings (e.g., Rain-X® Original Glass Treatment, made by SOPUS Products of Houston, Tex.) can be applied to the lens for enhanced visual clarity.

According to one embodiment, washing the output tip with water operates to clean the coated, or non-coated, intraocular camera lens. In output-tip washing or other lens cleaning embodiments and/or any other water (e.g., sterile water) embodiments described herein, a gelled water or viscoelastic gel (e.g., a viscous water based gel, such as viscasil®, available at www.viscasil.com), which can be transparent, may be used alone or in combination with water or other fluids or liquids. Any of the mentioned embodiments implementing fluid (e.g., water) for lens cleaning may incorporate any of the methods and structures described herein for adding fluid (e.g., water).

Tonometric techniques of depth measurement may comprise measuring pressure at a plurality (e.g. three or four) of locations on the meibomian gland before a procedure is initiated. Pressure measured during the procedure then may be interpreted according to the initial pressure, with the interpretation providing an estimate of depth. A similar method may be applied to techniques for depth measurement using electric fields, magnetic fields, and chemical sensing. Mechanically, a Q-tip multi-wavelength laser device may be employed to detect depth at a bottom of a cut. For example, one wavelength (i.e., color) may indicate depth; another color may indicate vascularization related to cancer growth. Black light may be useful in identifying whites, so one approach is to continue cutting until whites can no longer be seen. In other embodiments, a UV light may be placed for ease of use in determining the area to be treated while viewing the appropriate depth. Alternatively, if a wavelength is chosen that makes blue visible, then cutting may continue until a blue hue is Observed. Summarizing, different wavelengths of light may be sensitive to different characteristics of, for example, the meibomian gland. These differing sensitivities may be exploited to determine a condition of a tissue being treated (e.g., the meibomian gland) during a procedure, the condition being different at different layers of tissue.

Alternatively, a doctor may form a test perforation through the ocular surrounding tissue and into the meibomian gland (i.e. extract a core sample), the test providing an indication of liquefaction, and depth of the meibomian gland. This indication may be used to determine and refine a treatment procedure (i.e. type of ablation, number of ablations, their locations and depths). The amount of lipid formation in the meibomian gland may relate to the ability of the treatment to perform consistently. Meibomia in the tissue surrounding the orbit may relate to meibomian gland while colors may aid in identifying components of the tissue surrounding the orbit. A combination of the above tools including, in one example, an olfactory detector (e.g., sniffer), can be used to determine locations and appropriate times for performing a procedure. In certain embodiments, applied in addition to as an alternative to any of the above features, patterns of treatments can be determined by a device, which can mark and/or apply the treatments in areas based upon a liquefaction theory wherein the treatments are imparted into the meibomian gland (using, e.g., a scanning laser) in the determined areas.

In addition to pre-operative measurements of depths of the layer or layers being affected, depths of remaining tissue layers at the bottoms of treatments may be measured during formation of the treatments in real-time), with one or more operating parameters such as remaining treatment formation (e.g., cutting) time, pulse width, repetition rate, average power, coolant, etc., being adjusted in accordance with the results of the real-time depth measurement. For instance, a pre-operative scanning measurement may determine a meibomian gland to be about 700 microns, and ½ second into the formation of a treatment a real-time depth measurement may indicate a remaining depth of the meibomian gland at the bottom of the treatment being formed to be about 325 microns. It may be determined (e.g., automatically determined) at that time to continue formation of the treatment for another ½ second. This iterative process may be repeated, wherein for example a subsequent real-time measurement of remaining-depth of about 100 microns may be detected ¼ second later thus triggering, for example, a decision to continue formation for another ⅛ second, Various combinations and implementations of depth analysis, cutting type, speed control, and feedback algorithms, among other parameters, may be implemented in various combinations, for monitoring and controlling treatment formation depths and formation characteristics, for obtaining, among other things, one or more of greater monitoring control and treatment formation accuracy. For example, the laser may have a tip of 200 microns and enter the “treatment tissue” to a predetermined depth as seen by ultrasound technology, artemis technology, confocal microscopy, tonometry, laser, or UV light. The power will be in the range of 0.01 watts and the repetition rate of 10 Hz, but will vary with other manufacturer specifications for their device.

Also, when scanners are used, initial steps comprising, for example, determining one or more reference points of the eye (e.g., a center of the pupil, one or more points on the patient's retina, triangulated unique points on the patient's iris, and/or treatments or other markings formed on the patient's eye at an early stage of a procedure for the purpose of, for example, those treatments being used as reference points) may be implemented so that locations of treatments may be defined and/or recorded relative to the one or more reference points for use during the initial formation of the treatments and/or for use during follow-up procedure(s) wherein treatments may be modified and/or additional treatments may be formed. In accordance with one aspect, treatments formed during an initial or earlier procedure are used as reference points during remaining steps of the initial procedure and/or for the forming of additional treatments during follow-up procedures. For example, density mapping may be implemented wherein ultrasound is used to facilitate detection of tissue features such as a surface topography (e.g., locations of previously formed meibomian glands) for use as reference points. Also, depths of previously formed treatments may be detected to provide an option of, for example, augmenting depths of one or more treatments according to desired protocols. A topography unit will map the tissue surrounding the orbit and form a grid. The grid will be placed over the eye with the “treatment” sites marked and then lased or treated by a method of removing meibomian gland obstruction.

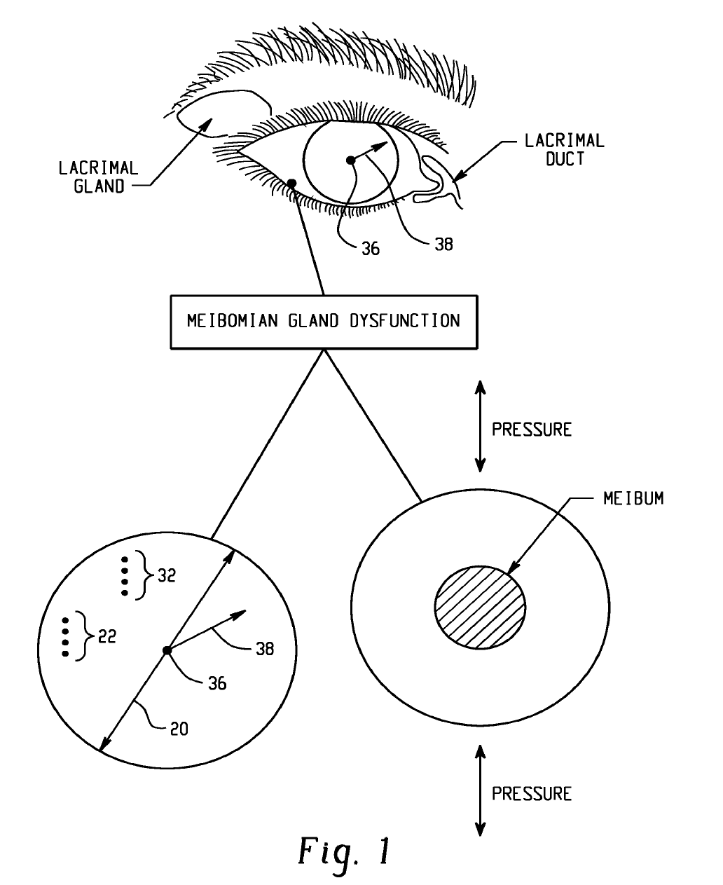

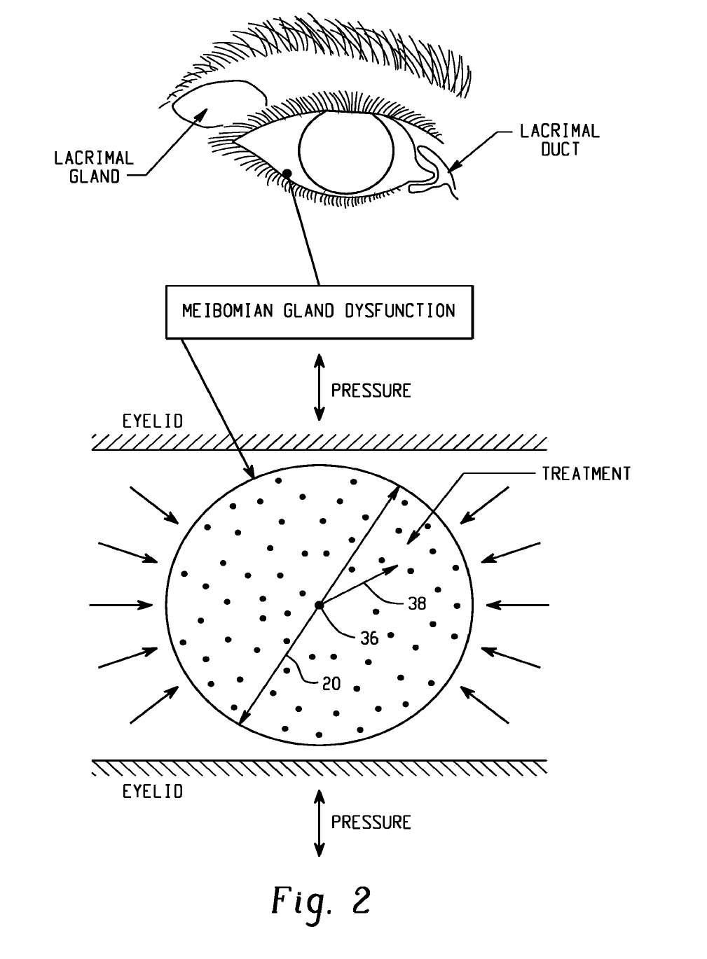

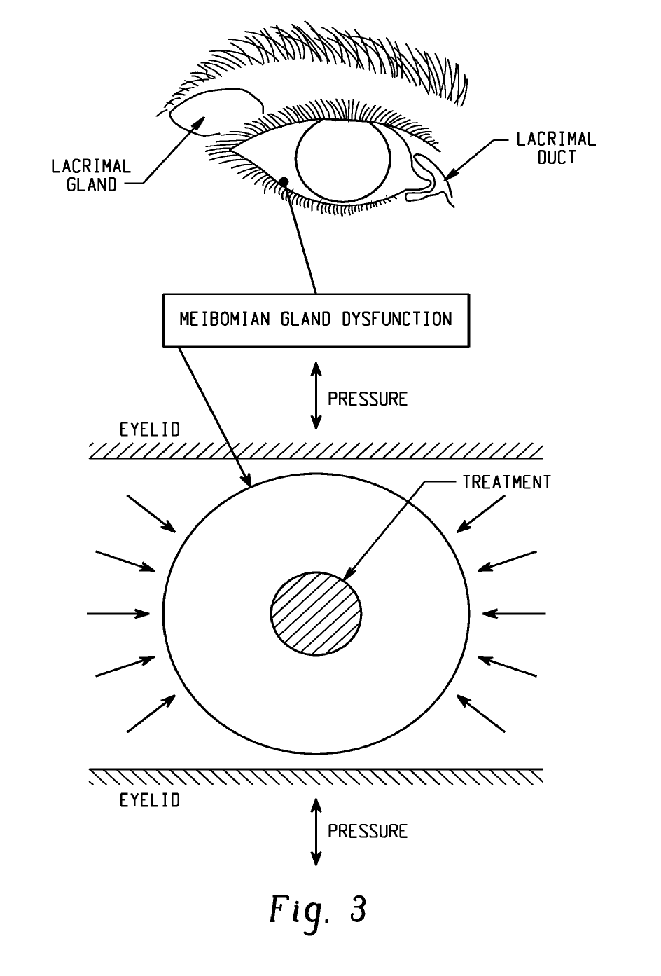

Referring more particularly to the drawings, FIG. 1 shows a schematic plan view of the right eye of a patient, and FIG. 2 is a side-elevation view of the eye depicted in FIG. 1. In accordance with an aspect of the present invention, treatments (e.g., groupings of treatments) may be applied to portions of, for example, surface areas of the meibomian gland disposed within the tissue surrounding the orbit. A few exemplary groupings of treatments, shown as point perforations in the illustrated examples, are shown in FIGS. 1 and 2, wherein the exemplary groupings are described in accordance with a polar coordinate system. Regarding the polar coordinate system, for reference, a center point 36 of the eye is designated as the pole and a line 38 is designated as the polar axis (e.g., zero degrees).

According to a more specific example, ablating optical energy can be focused using optics into the meibomian glands so that a peak concentration of the ablating optical energy occurs within the meibomian glands and a concentration of the optical energy in the tissue surrounding the orbit is substantially lower or, in one embodiment, below an ablation threshold. Dye enhancing the tissue to be treated can be used, for example, to facilitate one or more of assuring that the treatment energy (e.g., laser energy) penetrates the desired area wherein different colors of dye may be used, assuring that the treatment energy (e.g., laser energy) penetrates to the appropriate pre-determined depth wherein different consistencies and colorations can be used to this end, and allowing for better viewing of the treatment area. wherein dyes can be used in conjunction with the appropriate light source for “high lighting” and the background light can be reduced for enhancement. For example, the meibomian gland can be stained with yellow dye allowing for the location of diseased meibum (e.g., clogged meibomian glands) to be highlighted a darker yellow. In general, regarding dye enhancing of the tissue to be treated according to the present invention, dyes may typically be red, green or dark in nature and can be used to enhance the depth, length or width of the incision of the tissue to be treated. Such methods typically may be combined with treatment energies such as infrared energy. The operating parameter can vary depending on the type of enhancement used, type of tissue, desired depth, length and width, and the spectrum of energy used. Thus, in the context of, for instance, the preceding example, the term “non-invasively” should be interpreted to mean that portions of the meibomian gland penetrated by the treatment energy are not substantially affected (e.g., not ablated), or are affected to a lesser extent than that to which the underlying ocular tissue is affected, by the treatment energy.

As used herein, and not merely in the context of the present example, the term “invasively” should be interpreted to mean that portions of the tissue (e.g., meibomian glands and or any other tissues) penetrated by the treatment energy are substantially affected (e.g., ablated) by the treatment energy, Invasive penetration of tissue by treatment energy may generate, for example, a treatment.

In other examples, one or more of the treatments can be applied to penetrate through the tissue surrounding the orbit (e.g., to invasively penetrate wherein penetrated portions of the tissue surrounding the orbit are affected, such as by being ablated) and to treat (e.g., ablate) the meibomian gland. According to a particular implementation, a collimated beam of ablating optical energy may be directed through both the tissue surrounding the orbit and through, for example, a majority or more of the thickness of the meibomian gland, whereby tissues of both the tissue surrounding the orbit and meibomian glands are ablated along the path of the collimated beam. The parameter ranges can, in exemplary embodiments, be dependent upon desired, predetermined or expected wavelengths, lengths, widths and/or heights of incisions, and exemplary tissue parameters/types to be affected can include tissue surrounding the orbit and meibomian gland tissue. In certain implementations, the treatment energy beam can be shaped in the form of a complete treatment (e.g., elongated kerf). A mapping will determine the location, pattern, shape and landscape of the region acquiring the treatment based on density. The treatment energy beam can be completed by contact or non-contact of the laser energy in a pulse mode, or continuous mode that is proximal to the treatment area using a fiber based or scanner based delivery system with a predetermined software pattern or template. A beam splitter may be used to disperse energy of the beam in a pattern of the treatment area.

Dye-enhancing the tissue to be treated can, for example, be implemented. Dyes can comprise, for example, red, green or other relatively dark colors and can be used to enhance (e.g., selectively enhance by application to certain areas and/or selective coupling or matching of laser types to tissue and dye types) or otherwise affect the depth, length, width or other characteristic of the incision of the tissue to be treated. For instance, an area can be dyed for pretreatment with a laser having a wavelength that is substantially or highly absorbed by blood, wherein following (or during) the dying the heating laser energy can be directed over the dyed treatment areas to cause heat or to otherwise affect a propensity of such treatment areas to bleed during subsequent formation of the treatments. In certain embodiments, the treatment markings themselves may be formed as the dyed areas. In other embodiments, the depth, length, width or other characteristic of the incision of the tissue to be treated can be contacted with energy from a laser having a wavelength that is substantially or highly absorbed by blood, wherein following (or during) the contacting the heating laser energy can be directed over the treatment areas to cause heating or to otherwise affect a propensity of such treatment areas to bleed during subsequent formation of the treatments.

According to typical implementations, steps may be incorporated to ensure that pretreatment heating energy or subsequent ablating energy does not adversely affect the retina or other tissues, Such implementations may embody one or more of relatively low energy levels, tissues-type and/or color (using, e.g., dyes) matching with relatively high-absorption wavelengths (e.g., Nd:YAG or Er, Cr:YSGG), and focusing of the energies well in front of the retina.

Any one or more of the preceding methods may be practiced or combined with, for example, application of infrared energy as the treatment-energy, wherein, again, operating parameters can vary depending on one or more of the desired type of enhancement, type of tissue, depth, length, width, other characteristic, and spectrum of energy used.

A dimension (e.g., a cross-sectional shape or area measured in a direction transverse to a direction of propagation of the treatment energy) of a treatment may remain relatively constant through a depth of tissue (e.g., the tissue surrounding the orbit and/or meibomian gland) or may change with depth. For example, one or more treatments may be formed to have cross-sectional shapes or areas that decrease (or, alternatively, increase) with depth into the meibomian gland, such as would be the case, for example, with a circular treatment having a diameter that decreases with increasing depth into the meibomian gland. In typical implementations, a treatment (e.g., a conically-shaped treatment according to the preceding example) may comprise, for example, a diameter that tapers from about 0.1 to about 100 percent with each 1 percent drop in depth. In a particular example, the diameter may drop by about 1 percent for each 1 to 20 percent drop in depth. In the context of for example, a tissue implant (e.g., a conically-shaped tissue implant) being formed in the meibomian gland, by way of treatment energy being directed non-invasively through the tissue surrounding the orbit, a tissue implant dimension (e.g., diameter) may taper within the meibomian gland from about 1 to about 100 percent with each 1 percent drop in depth and, in a particular example, may drop by about 1 to about 20 percent for each 1 percent drop in depth within the meibomian gland.

Removed or affected areas corresponding to treatments may for example be filled-in by a surgeon with any known biocompatible materials, such as, for example, Tisseal, anti-inflammatories or antibiotics. In accordance with one aspect of the invention, removed or affected areas corresponding to treatments are at least partially filled-in by the body (e.g., via the body's natural response) with sub-meibomian glandular tissue which may, for example, augment a property of the eye. For example, in the case of the meibomian gland, the new sub-meibomian glandular lipid-based tissue infiltrating a removed or affected area of the meibomian gland may have a greater elasticity or be more flexible than the original tissue surrounding the orbit. The body's introduction of healthy meibum into removed or affected areas thus may increase the viscosity of, for example, one or more of the meibomian glands secretions of meibum. In the example of removed or affected areas in the tissue surrounding the orbit, new sub-glandular tissue in, for example, the meibomian gland may facilitate or enhance a functionality or other property of the underlying tissue surrounding the orbit.

According to typical implementations, the meibomian gland may be treated by directing treatment energy through the over the tissue surrounding the orbit with use of laser technology, whereby as previously mentioned the meibomian gland may be treated with treatment energy (e.g., laser energy) aimed (e.g., focused) in the tissue surrounding the orbit, leaving the adjacent structures relatively undisrupted, For example, laser energy can be directed to focus or converge on the underlying meibomian gland wherein, for example, the laser energy has a relatively low power density (e.g., a large spot size) on the tissue surrounding the orbit while at the same time having a relatively high power density (e.g., a relatively small spot size) on the underlying meibomian gland, and wherein the absorption rate is that of meibum lipids so that the laser energy forms a “v” in the meibomian gland that focuses to dose (e.g., cut) only the meibomian glandular tissue and/or adjacent or immediately-adjacent matter (e.g., obstructing and/or non-eye material such as material comprising meibum lipids and/or lipid deposits). As will be discussed below, the tissue surrounding the orbit may be rotated or torqued from a different site at varying degrees in order to obtain, for example, better cosmetic effects (e.g., reduced reddening). Treatments (e.g., cuts or kerfs) employed in such procedures may be formed in varying shapes as previously mentioned. Typical shapes can include, as examples, “u” and “v” shapes. The kerfs may also be made wherein the center of the kerf has more tissue than the edges. Generally, a kerf can have a width that varies according to different density factors and meibum in different meibomian glands. However, incisional meibomian depths of treatments that are greater than 90% may, in certain implementations, remain constant. According to certain embodiments, an ultrasound unit can be used to remove both meibum and lipidous tissue. In other embodiments, cautery can be used, for example, to improve the clarity of the site where treatments are to be formed and/or to generate the treatments. Moreover, a light having a certain color, such as a black light, may be used to enhance a view of tissue surrounding the orbital tissue in certain embodiments. Further, various colors may be placed in a scope (e.g., microscope) to enhance vision (e.g., surgeon discernment of features). For instance, green may allow a user to better see depth of penetration. Additionally, a tonometer may be used to detect pressure of a treatment area, and/or a femtosecond laser can be used to remove or cut tissue of the treatment.

One or more of the treatments may be introduced with the adjacent structures in place, wherein for example the tissue surrounding the orbit is left in a naturally-occurring orientation over the meibomian gland. In such embodiments, penetration paths through/into the meibomian gland and meibomum may be aligned or substantially aligned. For example, a beam of electromagnetic energy may be directed through both the undisturbed meibum and through, for example, a majority or more of the thickness of the tissue surrounding the orbit. The beam may travel through the tissue surrounding the orbit in a non-invasive or invasive manner as described above, whereby, in the latter case for example, tissues of both the meibomian gland and tissue surrounding the orbit may be ablated along the path of the beam of electromagnetic energy.

One or more of the treatments described herein may be introduced with parts or substantially all of the tissue surrounding the orbit altered (e.g., removed, reconfigured or repositioned such as by rotating the tissue, or separating and/or shifting the meibomian relative to the meibum) before or during introduction of the one or more of the treatments, in any order or sequence of steps. Thus, with any of the implementations described herein, parts of the tissue surrounding the orbit may, in certain embodiments, be manipulated while other parts are left in a naturally-occurring orientation over the meibomian gland. In other implementations, parts of the tissue surrounding the orbit above portions of the eyelid receiving treatments may be manipulated and/or other parts of the meibomian gland above portions of the eyelid receiving treatments may be left in a naturally-occurring orientation over the meibomian gland.

Furthermore, with any of the implementations described herein, substantially all of the meibomian gland may be reconfigured Or repositioned (e.g., shifted or rotated about center point 36) relative to, for example, the tissue surrounding the orbit.

Moreover, in addition, or as an alternative, to the present invention's altering of the meibum before or during application of treatments, other aspects of the present invention may comprise introducing one or more of the treatments through the eyelid in one or more of the pre- or post-altered states of the lipids. With respect to exemplary embodiments wherein the meibomian gland is repositioned before application of treatment energy and formation of treatments, once the meibomian gland is brought to (or brought back to) assume (or at least to approximate) a naturally-occurring configuration or orientation (or is otherwise brought to a post-treatment configuration or orientation), some or all of the penetration paths through/into the meibomian gland and meibum are not aligned. This lack of alignment between penetration paths of the tissue surrounding the orbit and meibomian gland, or alternatively the covering-up of penetration paths through the eyelid in embodiments wherein, for example, penetration paths are not formed in part or all of the tissue surrounding the orbit, can serve to provide, for example, one or more of a sealing effect for enhanced healing and structural integrity to the affected layers.

With reference again to FIG. 1, an example of repositioning the tissue surrounding the orbit can include rotating the tissue surrounding the orbit, relative to the meibomian gland, before application of the treatments. The tissue surrounding the orbit can be gripped and rotated an amount, such as, for example about 1 to 2 degrees, or more broadly about 1 to 90 degrees. In other implementations, the rotation may range from about 1 to about 45 degrees, or more, and/or different portions of the tissue surrounding the orbit may be rotated, for example, at different points in time, in different directions and/or in different amounts. Following such rotation, the tissue surrounding the orbit may (or may not) be held in the rotated position, for example, while some or all of the treatments are applied. After application of some or all of the treatments, the tissue surrounding the orbit can be moved back, to a full or partial extent, to its naturally-occurring orientation and/or can be released so that the tissue surrounding the orbit moves, to a full or partial extent, back to its naturally-occurring orientation.

In other implementations, after application of some or all of the treatments, the tissue surrounding the orbit can be rotated in the opposite direction to a greater extent than that to which it was first rotated, such as rotation in the counter-clockwise direction about 1 up to 90 degrees. Following any of the rotations or shifts of the tissue surrounding the orbit described herein, and/or at any intermediate step, part or all of the tissue surrounding the orbit being altered may be held using any known temporary or permanent means.

In further implementations, after application of some or all of the treatments, the tissue surrounding the orbit can be rotated in the opposite direction to a greater extent than that to which it was first rotated, such as rotation in the counter-clockwise direction about up to 90 degrees. Following any of the rotations or shifts of the meibomian gland described herein, and/or at any intermediate step, part or all of the tissue surrounding the orbit being altered may be held with any known temporary or permanent means as previously mentioned.

In other implementations, following an initial rotation of the tissue surrounding the orbit, application of one or more treatments (e.g., a treatment in the shape of a radially-extending spot or a row of treatments forming the spot) can be made through one or more treatments (e.g., elongate kerf(s) or doses of energy) in the meibomian gland. The tissue surrounding the orbit can then be rotated in the same direction to a greater extent than that to which it was first rotated. Then, one or more treatments (e.g., a treatment in the shape of a radially-extending spot or a row of treatments forming the spot) can again be formed in the tissue surrounding the orbit through the same treatments already formed in the tissue surrounding the orbit so that the meibomian gland is minimally impacted. The process can be repeated to form additional treatments of, for example, the same shape in the meibomian gland, through the same treatments already formed in the tissue surrounding the orbit. In this example, the tissue surrounding the orbit is progressively rotated in one direction with treatments being formed through the same opening(s) in the tissue surrounding the orbit at each step. In modified embodiments, the tissue surrounding the orbit can be rotated in the opposite direction (e.g., past the original, naturally-occurring orientation) to various degrees to facilitate formation of one or more treatments (e.g., a treatment in the shape of a radially-extending spot or a row of treatments forming the spot) in the tissue surrounding the orbit through the same treatments already formed in the meibomian gland so that the meibomian gland is minimally impacted again. Accordingly, the meibomian gland can be rotated in both directions to facilitate formation of various treatments in the tissue surrounding the orbit, all through the same opening (e.g., treatment) in the meibomian gland. As a result of the reduced number of treatments being formed in the meibomian gland, redness and/or heating time can be attenuated or eliminated.

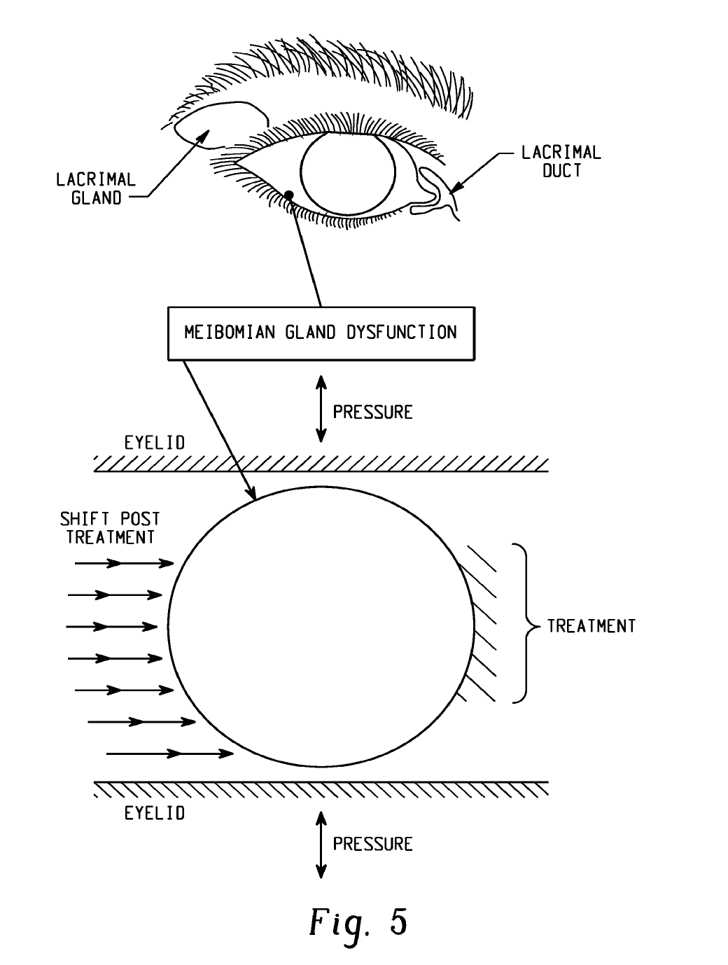

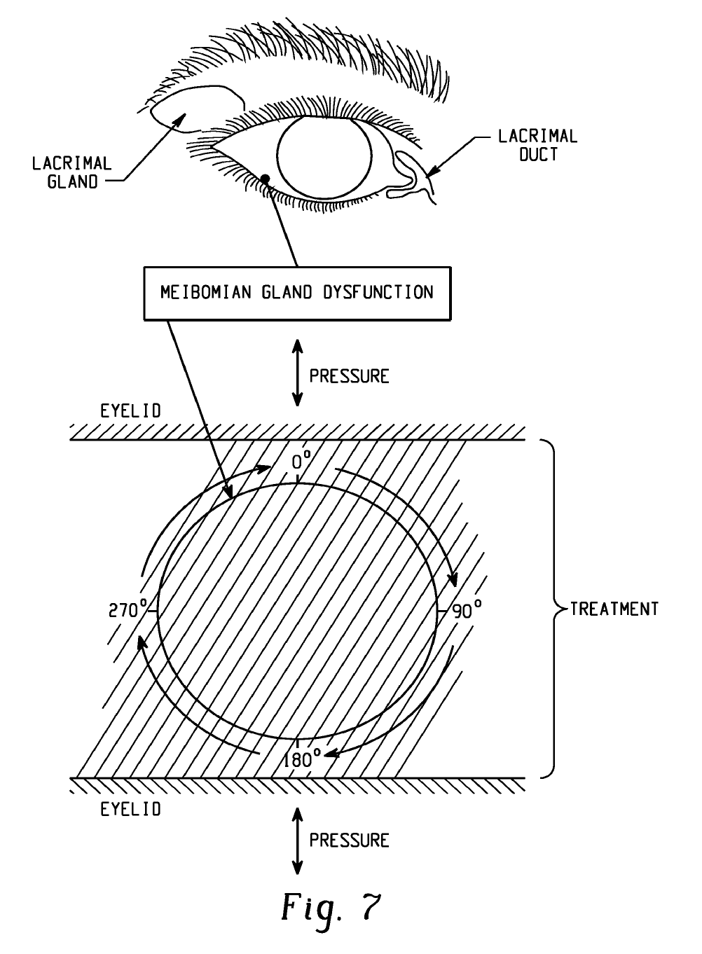

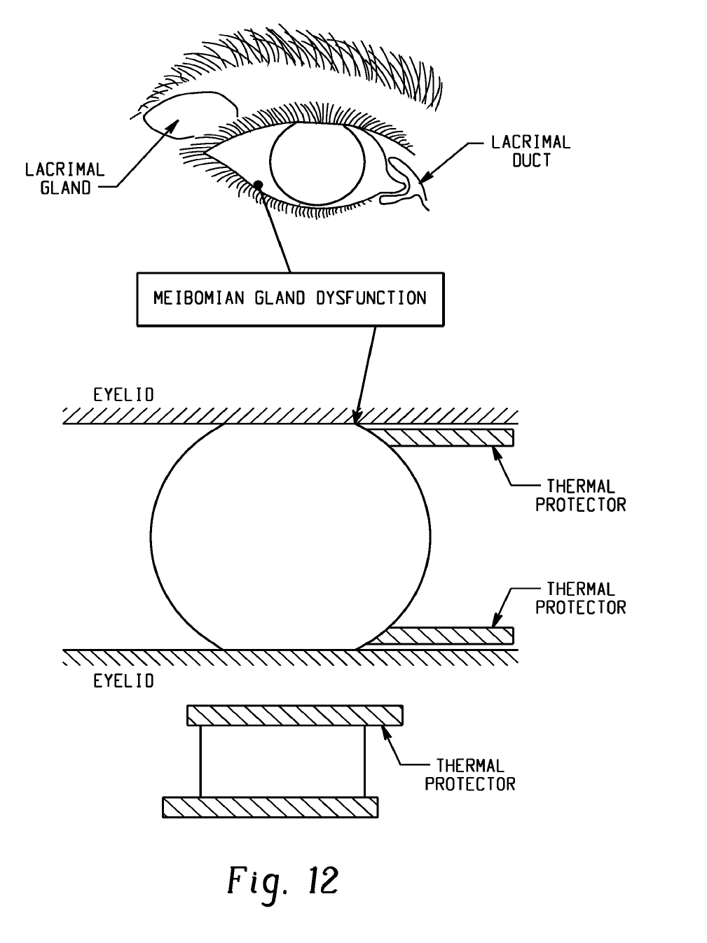

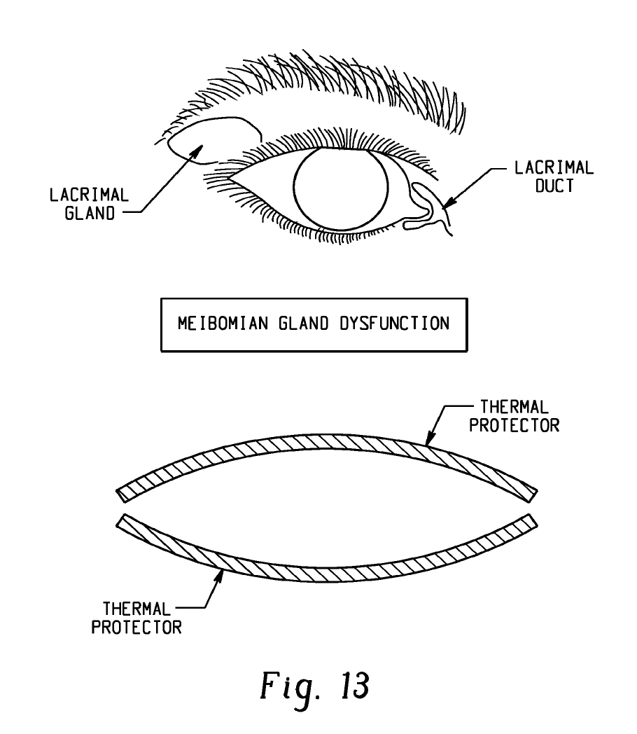

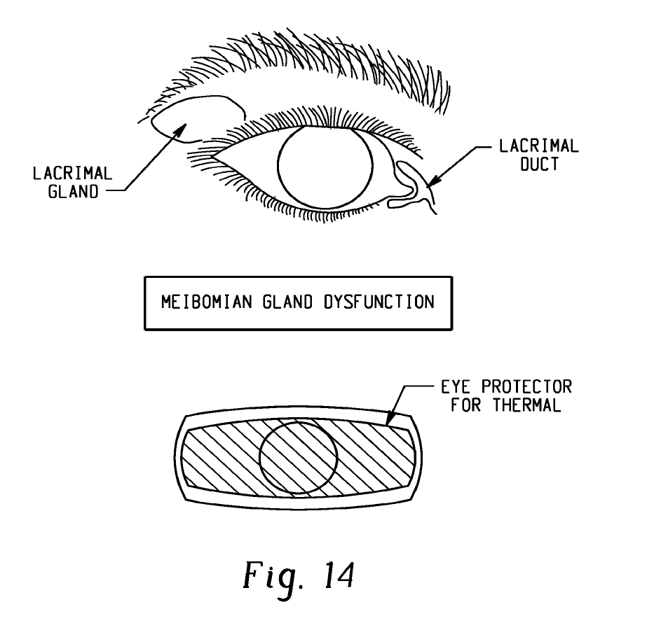

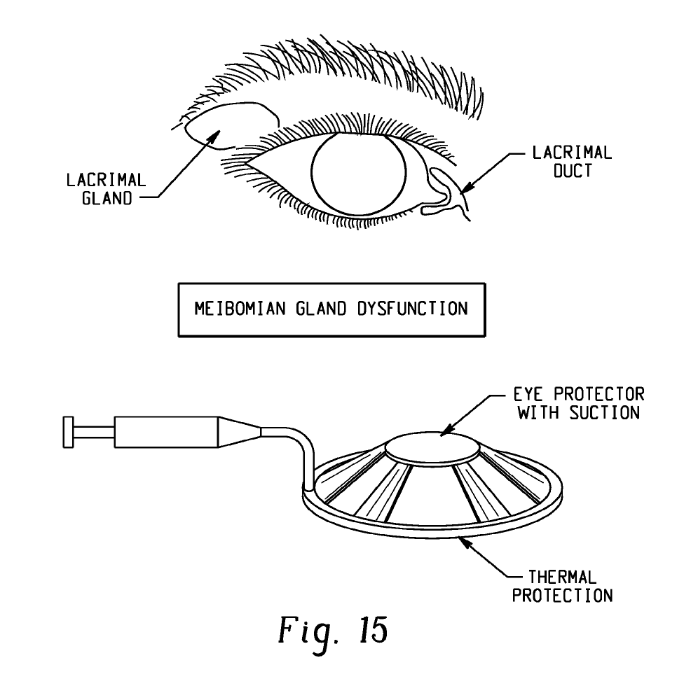

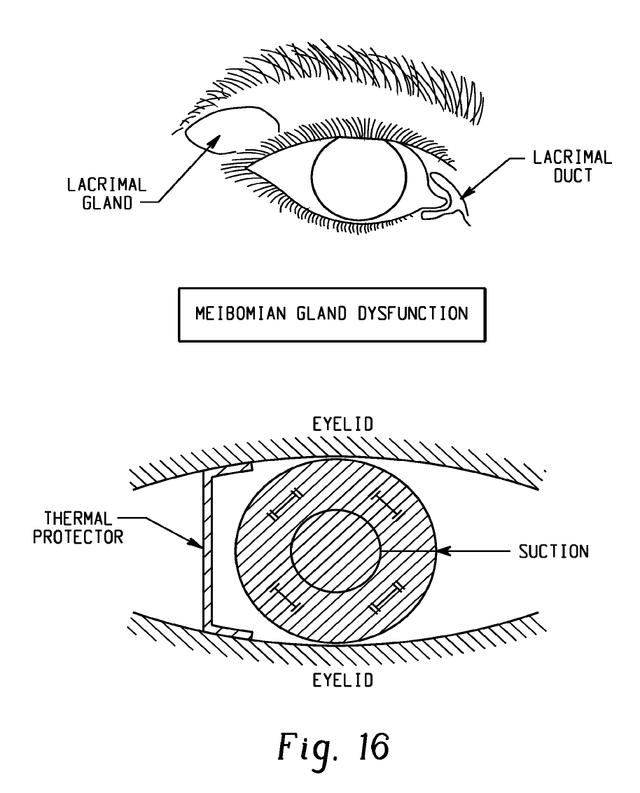

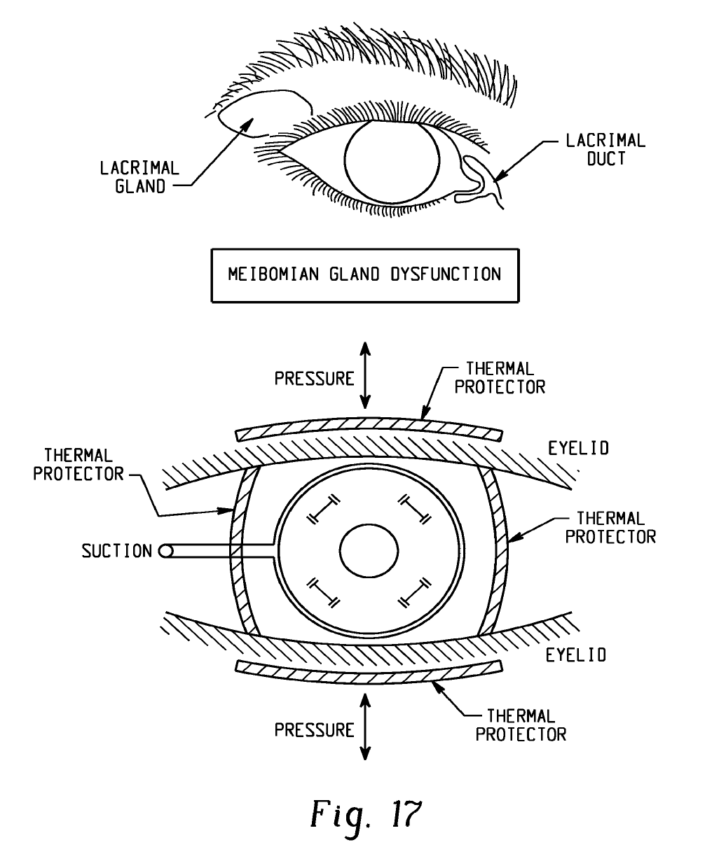

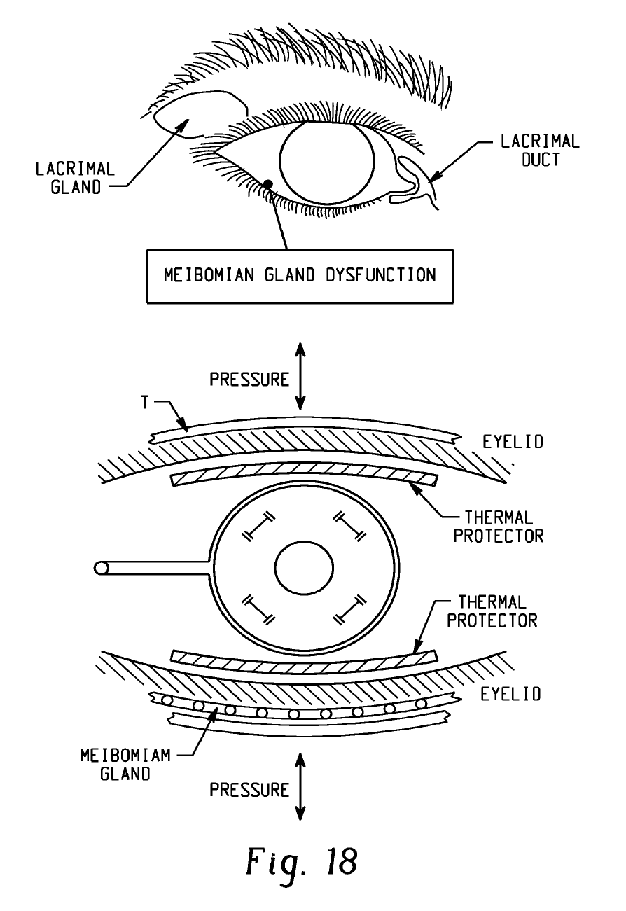

FIGS. 5-14 illustrate various implementations of methods for repositioning (e.g., rotating) the meibomian gland relative to the tissue surrounding the orbit. The treatments in the meibomian gland and/or tissue surrounding the orbit can comprise, for example, elongated or spot-shaped treatments such as those shown in the present examples of FIGS. 5-14, and/or may comprise groupings of treatments as discussed in any of the previously-mentioned examples, or combinations and permutations thereof, in various positions, shapes and patterns (e.g., fewer or greater numbers of elongated treatments, of the same or different lengths as those shown, at for example one or more of 0, 90, 180, and 270 degrees). For instance, one or more (e.g., each) of the shown treatment elongated shapes may comprise, instead of an elongated kerf as shown, a series of smaller treatments forming the same general shape. Moreover, one or more of the treatments in the meibomian gland may comprise varying (e.g., reduced) sizes relative to the corresponding treatments formed therebeneath in the tissue surrounding the orbit, as elucidated in the illustrated examples of FIGS. 7-10, 12 and 14.

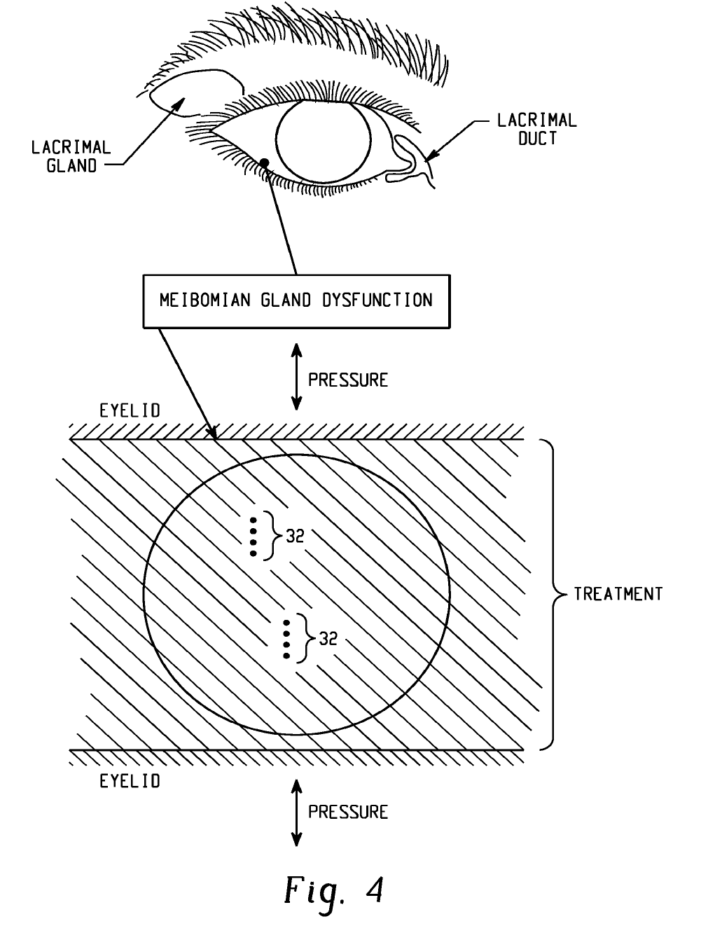

FIG. 4 illustrates a spot size encompassing the meibomian gland and tissue surrounding the meibomian gland. Pressure, vibration, rotation or shifting may then be used to apply pressure tangent to the meibomian gland to increase meibum liquefaction excretion.

With particular reference to FIG. 5, this sequence depicts a rotation process wherein treatments are marked, for example, at 0, 90, 180, and 270 degrees. In FIG. 5, locations for formation of treatments are marked on the meibomian gland, and in FIG. 5 the meibomian gland is moved (e.g., rotated or torqued) or shifted in some way or to some degree, The meibomian gland can, for example, be contacted (e.g., gripped) using a meibomian gland template device and moved.

FIG. 5 shows that treatments can then be formed in both the meibomian gland and tissue surrounding the orbit at locations corresponding to the post-movement positions of the markings, and in FIG. 5 the meibomian gland can once again be moved (e.g., rotated, torqued and/or shifted) in some way or to some degree. For example, the meibomian gland can be moved (e.g., rotated, torqued and/or shifted) in some way or to some degree so that the treatments formed in the tissue surrounding the orbit are at least partially, and in certain embodiments, completely, covered by non-treatment areas of the meibomian gland. According to certain embodiments, the meibomian gland can be moved back (to the same, lesser or greater extent) in a direction from which it was first moved, but in modified embodiments it may be moved at least in part (to the same, lesser or greater extent) in other directions. As presently embodied, the meibomian gland can be rotated so that the angular locations of the markings are changed from their post-movement angular positions, and in the illustrated example of FIG. 5 the meibomian gland is rotated so that angular locations of the markings are changed back to locations corresponding to the pre-movement positions of the markings corresponding for example to the naturally-occurring orientation of the meibomian gland. The meibomian gland can be moved using for example the meibomian gland template device. Following any of the movements of the meibomian gland described herein, and/or at any intermediate step, part or all of the meibomian gland being altered may be held with any herein-described or known temporary or permanent means, such as the meibomian gland template device.

In certain embodiments, fluids, including water, sterile water or conditioned fluids, such as described in U.S. Pat. Nos. 5,785,521 and 6,350,123, the contents of which are incorporated herein by reference, may be added to ensure or aid in the cosmetic appeal of the treated tissue and/or to assist with healing time Or other properties. For example, fluid (e.g., sterile water) may be applied by way of a small air mister (e.g., from a local or remotely-disposed canister or dropper) affixed, for example, to a device (e.g., an applinator device or output tip), between or, preferably, during application of treatment energies, to thereby attenuate or eliminate charring and/or wash away blood. As another example, fluid (e.g., sterile water) may be applied by way of a small air mister or sprayer spot affixed, for example, to a treatment energy (e.g., laser) device (e.g., handpiece) at or for any of the above-noted times or purposes. The spot may comprise, for example, tubing (e.g., clip-on and/or silicone based tubing) secured to an outside or built into the device and a fluid dispensing input disposed on the device. The fluid-dispensing input may be activated, for example, to facilitate manual or powered dispensation of fluid. Manual dispensation may be implemented by way of, for example, a spot leading to or integrally formed with a detachable container (e.g., pod) that can be squeezed by a user to dispense fluid (e.g., sterile water pre-packaged into a single-use, disposable pod), and powered dispensation may be implemented by way of a toggle button to initiate a powered output of fluid at, for example, a relatively low flow rate and pressure. An atomized distribution of fluid (e.g., sterile water) particles may be automatically applied to the target during application of treatment energies, for example. In other examples, a drop of the fluid (e.g., sterile water) may be applied before or during application of treatment energies. In still further embodiments, treatment energies and fluid (e.g., sterile water) may be combined to facilitate electromagnetically induced mechanical cutting, as described in the preceding two patents, to enhance cutting attributes. Suction may be applied to any of the foregoing implementations, as welt, for removing fluids, debris and/or liquids. For any embodiments employing suction for any purpose described herein, such as to secure a structure to a surface of the eye, specialized surfaces (e.g., relatively nonporous surfaces to facilitate suctional gripping and securement of the structure to the eye) and/or surface treatments (e.g., the above-mentioned viscasil®) can be employed.

As shown in FIG. 5, treatments in the meibomian gland may be performed using techniques such as a pre-treatment shift causing treatment to be performed on a new casing of tissue whereby when the casing is released a new casing will be covering the treatment site. Sutures, surgical tacks, screws or staples, and/or applinator-style attachments including adhesives may be used for closure.

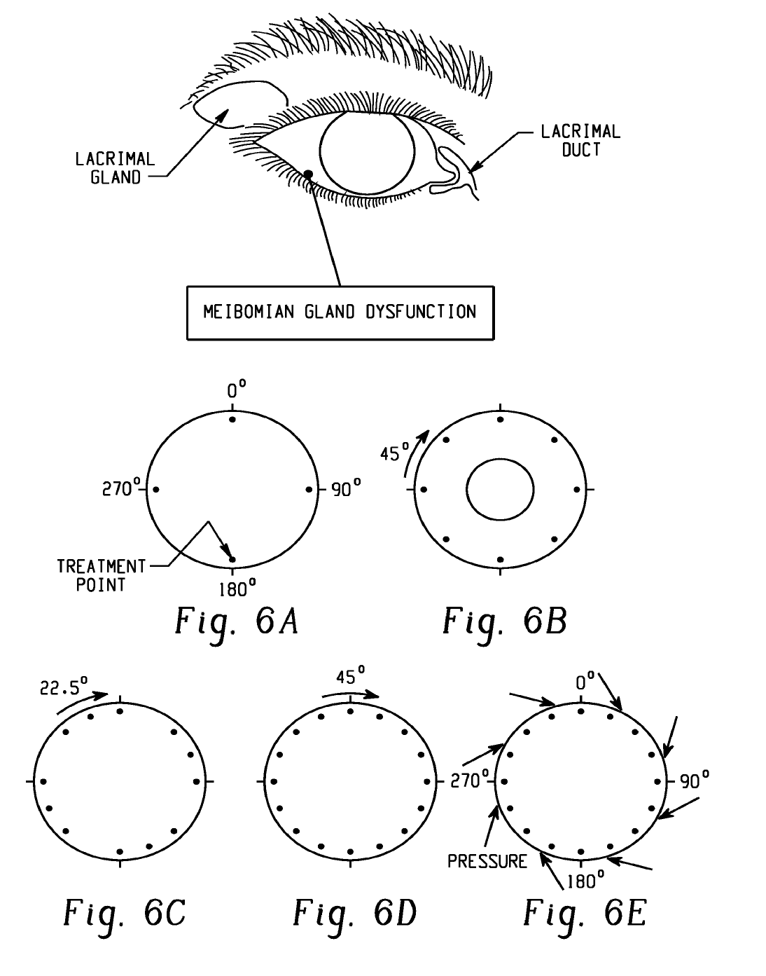

Referring to FIGS. 6a-6e, a rotation process is shown wherein treatment markings are formed on the meibomian gland at the exemplary locations of zero, ninety, one hundred and eighty, and two hundred and seventy degrees. As depicted in FIG. 6a, the locations for generation of treatments can be disposed on the meibomian gland in sets (e.g., pairs). One or more (e.g., all) of the sets can comprise, for example, a plurality of treatments or treatment groupings as described above, wherein the treatments or treatment groupings of one or more of the sets are configured to allow interweaving with one or more of the subsequently formed treatments or treatment groupings in the tissue surrounding the orbit. In the illustrated embodiment, the treatments or treatment groupings of the sets allow interweaving with the subsequently formed treatments or treatment groupings in the tissue surrounding the orbit (cf. FIG. 6d, infra). As presently shown, the treatments or treatment groupings of each set are spaced one from the other at different (e.g., greater) distances than for example those shown in FIG. 5.

In FIG. 6b the meibomian gland is moved (e.g., rotated or torqued) or shifted in some way or to some degree as described above. The meibomian gland can for example be contacted (e.g., gripped) using a meibomian gland template device and moved as described above. The meibomian gland can be rotated so that angular locations of the markings are changed from their pre-movement marked angular positions and, as presently illustrated, so that the post-movement angular location(s) of at least one of the markings of each set is disposed between two of the pre-movement locations of the markings of a corresponding set. According to the implementation illustrated in FIG. 6b, the post-movement angular location one of the markings of each set is disposed between two of the pre-movement marking locations of the corresponding set. In FIG. 6c the treatments can be formed in both the meibomian gland and tissue surrounding the orbit at locations corresponding to the post-movement positions of the markings as described above, and in FIG. 6d the meibomian gland can be moved as described above and the treatments in the meibomian gland closed as discussed above and depicted in FIG. 5. Modified embodiments similar to those discussed above in connection with FIG. 5 may be implemented, as well.

Referring to FIG. 7, a rotation process is shown wherein treatment markings are formed on the meibomian gland at the exemplary locations of zero, ninety, one hundred and eighty, and two hundred and seventy degrees. As depicted in FIG. 7, the locations for generation of treatments can be disposed on the meibomian gland in sets (e.g., pairs). One or more (e.g., of the sets can comprise, for example, a plurality of treatments or treatment groupings as described above, wherein the treatment markings (and/or treatments) in the meibomian gland comprise reduced sizes relative to the corresponding treatment markings (and/or treatments) of, for example, FIG. 1. According to another aspect, the treatment markings (and/or treatments) in the meibomian gland comprise reduced sizes relative to corresponding treatments that will be formed therebeneath in the tissue surrounding the orbit, as elucidated in the illustrated examples of FIGS. 7-10, 12 and 14. In the illustrated embodiment, each treatment marking (and/or treatment) comprises a single spot shape disposed at each angular location (e.g., each post-movement angular location) where a corresponding treatment or treatment grouping will be formed in the tissue surrounding the orbit.

In FIG. 7 the meibomian gland is moved (e.g., rotated or torqued) or shifted in some way or to some degree as described above. The meibomian gland can for example be contacted (e.g., gripped) using a meibomian gland template device and moved as described above. The meibomian gland can be rotated so that angular locations of the markings are changed from their pre-movement marked angular positions. In FIG. 7 the treatments can be formed in both the meibomian gland and tissue surrounding the orbit at locations corresponding to the post-movement positions of the markings as described above, and in FIG. 7 the meibomian gland can be moved as described above. Subsequently, the treatments in the meibomian gland can be closed as discussed above. Modified embodiments similar to those discussed above in connection with FIG. 5 may be implemented, as well.

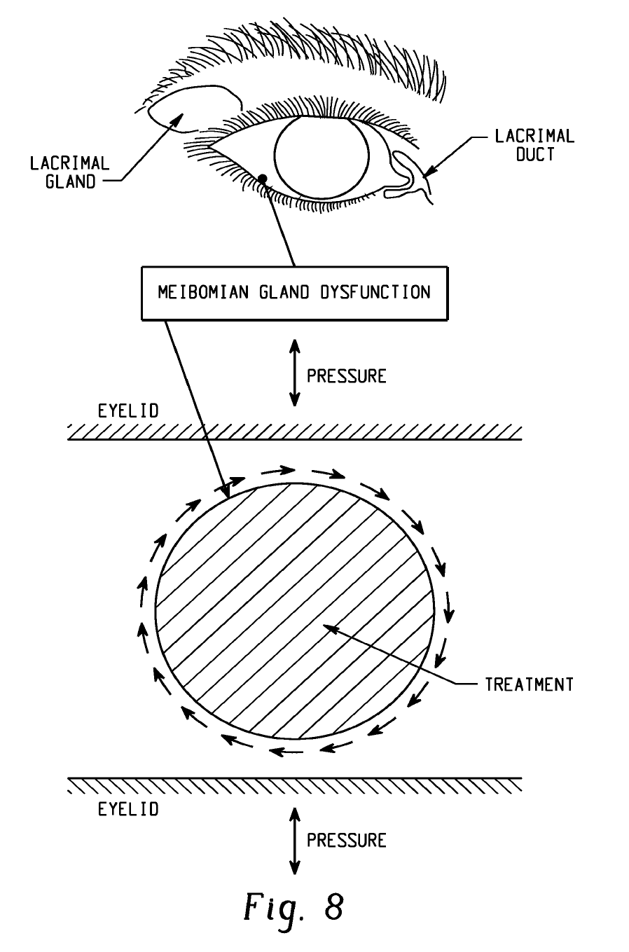

FIG. 8 depicts a particular implementation of the process of FIG. 7, wherein a pair of treatment markings is formed on the meibomian gland at zero, ninety, one hundred and eighty, and two hundred and seventy degrees. In FIG. 8, the meibomian gland is rotated or torqued in the clockwise direction about twenty to thirty degrees. In FIG. 8 the treatments are formed in both the meibomian gland and tissue surrounding the orbit at locations corresponding to the post-movement positions of the markings as described above, wherein the treatments in the meibomian gland comprise doses of energy disposed at each angular location (e.g., each post-movement angular location) and corresponding treatments in the underlying tissue surrounding the orbit comprise elongated shapes (e.g., elongated kerfs) extending radially outwardly at constant or substantially constant angular positions. In FIG. 8 the meibomian gland is rotated or torqued in a counter-clockwise direction twenty to thirty degrees back to its naturally-occurring orientation, followed by the treatments in the meibomian gland being closed as discussed above.

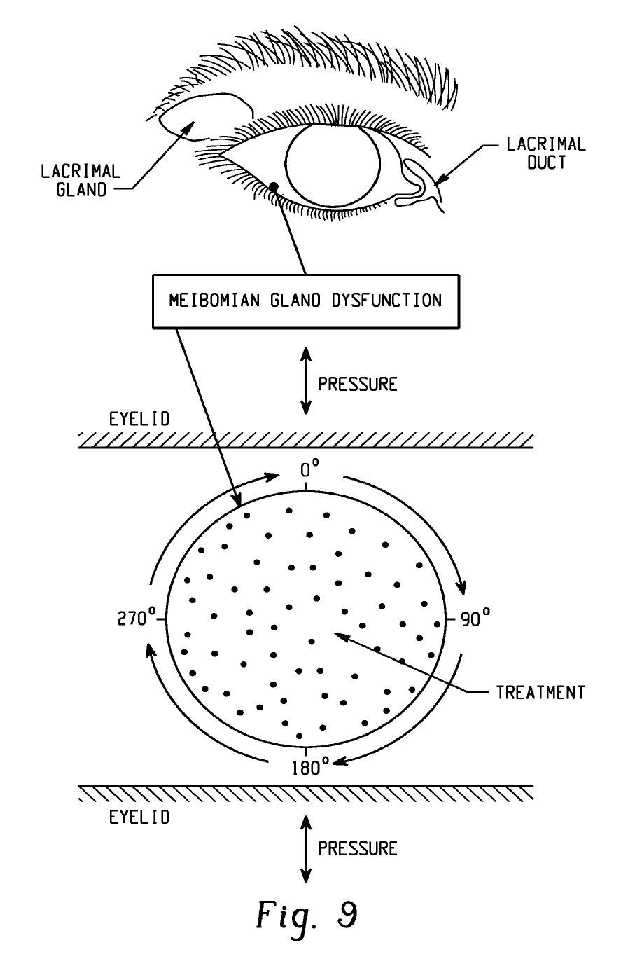

With reference to FIG. 9, a rotation process is shown wherein treatment markings are formed on the meibomian gland at exemplary locations of zero, ninety, one hundred and eighty, and two hundred and seventy degrees. As depicted in FIG. 9, the locations for generation of treatments can be disposed on the meibomian gland in sets (e.g., pairs). One or more (e.g., all) of the sets can comprise, for example, a plurality of treatments or treatment groupings as described above. Similarly to the embodiment of FIG. 7, the treatment markings (and/or treatments) on or in the meibomian gland comprise reduced sizes relative to the corresponding treatment markings (and/or treatments) of, for example, FIG. 1. According to one aspect, the treatment markings (and/or treatments) in the meibomian gland comprise reduced sizes relative to corresponding treatments that will be formed there beneath in the tissue surrounding the orbit. As presently shown, markings for the treatments or treatment groupings of each set are spaced one from the other at different (e.g., greater) distances than for example those shown in FIG. 5. In the illustrated embodiment, the treatment markings comprise spot shapes disposed at each angular location (e.g., each post-movement angular location) where a corresponding treatment or treatment grouping will be formed in the tissue surrounding the orbit. Furthermore, in exemplary embodiments markings for the treatments or treatment groupings of one or more of the sets are configured to allow interweaving of corresponding treatments or treatment groupings in the meibomian gland with one or more of the subsequently formed treatments or treatment groupings in the tissue surrounding the orbit. In the illustrated embodiment, markings for the treatments or treatment groupings of each set allow interweaving of treatments or treatment groupings in the meibomian gland with each of the subsequently formed treatments or treatment groupings in the tissue surrounding the orbit (cf. FIG. 9, infra).

In FIG. 9 the meibomian gland is moved (e.g., rotated or torqued) or shifted in some way or to some degree as described above. The meibomian gland can for example be contacted (e.g., gripped) using a meibomian gland template device and moved as described above. The meibomian gland can be rotated so that angular locations of the markings are changed from their pre-movement marked angular positions and as presently illustrated, so that the post-movement angular location(s) of at least one of the markings of each set is disposed between two of the pre-movement locations of the markings of a corresponding set. According to the implementation illustrated in FIG. 9, the post-movement angular location of one or more of the markings of each set is disposed between two of the pre-movement marking locations of the corresponding set. In FIG. 9 the treatments can be formed in both the meibomian gland and tissue surrounding the orbit at locations corresponding to the post-movement positions of the markings as described above. The treatments or treatment groupings can be formed in the meibomian gland to have reduced sizes relative to the corresponding treatments or treatment groupings in the underlying tissue surrounding the orbit. As presently embodied, the treatments or treatment groupings formed in the meibomian gland comprise reduced sizes (e.g., doses of energy) and the treatments or treatment groupings in the underlying tissue surrounding the orbit comprise elongated shapes (e.g., elongated kerfs) extending radially outwardly at constant or substantially constant angular positions. In FIG. 9 the meibomian gland can be moved (e.g., moved back) as described above, after which the treatments in the meibomian gland can be closed as discussed above. Modifications may be implemented similar to those discussed above in connection with FIG. 5.

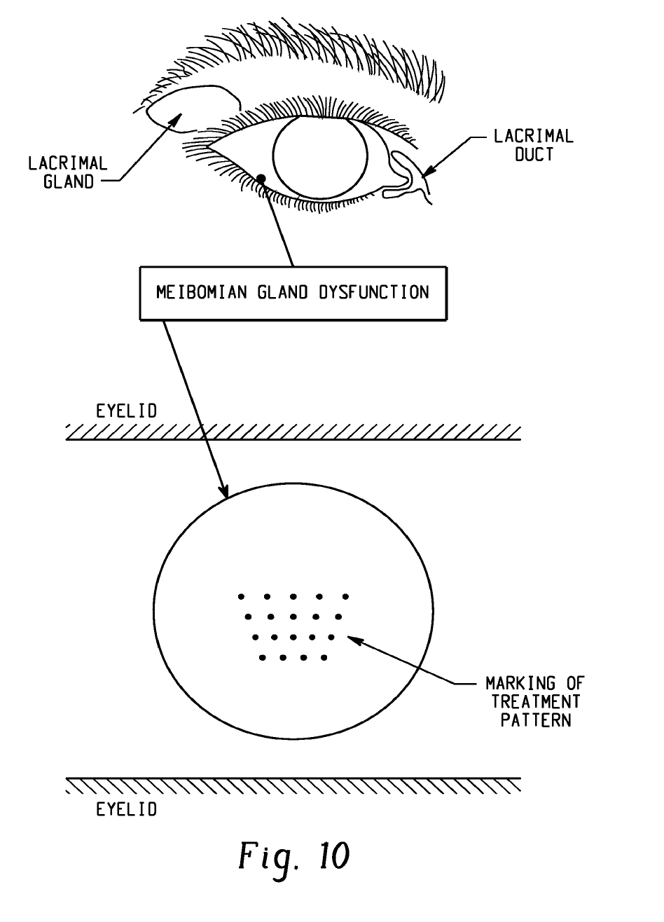

FIG. 10 depict a particular implementation of the process of FIG. 9, wherein a pair of treatment markings is formed on the meibomian gland at zero, ninety, one hundred and eighty, and two hundred and seventy degrees. In the implementation depicted in FIG. 10, a diameter of the cornea is about 16 mm and the treatment markings of each pair are spaced about 4 microns apart. In FIG. 10 the meibomian gland is rotated or torqued in the clockwise direction about seven to twelve degrees, so that following the procedure treatments in the meibomian gland will be interweaved with subsequently formed treatments in the tissue surrounding the orbit and the treatments in the tissue surrounding the orbit will not be exposed.

In FIG. 10 the treatments are formed in both the meibomian gland and tissue surrounding the orbit at locations corresponding to the post-movement positions of the markings as described above, wherein the treatments in the meibomian gland comprise doses of energy and corresponding treatments in the underlying tissue surrounding the orbit comprise elongated shapes (e.g., elongated kerf) extending radially outwardly. In the illustrated embodiment, the treatments of each pair in the tissue surrounding the orbit have widths of about 2 mm and are spaced about 2 mm apart. In FIG. 10 the meibomian gland is rotated or torqued in a counter-clockwise direction seven to twelve degrees back to its naturally-occurring orientation, followed by the treatments in the meibomian gland being closed as discussed above.

FIG. 11 is a view of the eyelid protected by a mechanism for thermal absorption so that the treatment is performed and not damage the eyelids.

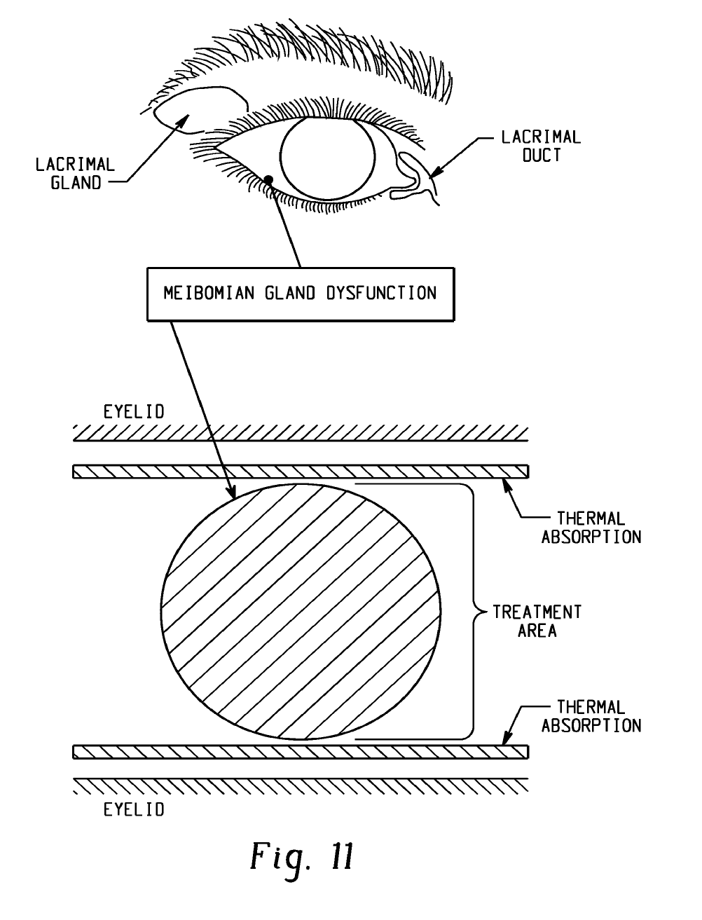

FIG. 12 is a drawing defining a thermal protector in relation to the eyelid and/or tissue being treated. Regarding the spot-shaped treatment markings (and/or treatments) on (in) the meibomian gland, the sizes and shapes of these items can be formed, for example, to be as small as possible while still enabling, for example, formation of corresponding treatments or treatment groupings there beneath in the tissue surrounding the orbit. In the illustrated embodiment, the treatment markings on and treatments in the meibomian gland comprise circular shapes approximating the cross-section of (e.g., and formed by) a fiber optic tip that can, in the illustrated embodiment, be used to form the treatments in the underlying tissue surrounding the orbit.

Formation of treatments in the meibomian gland and tissue surrounding the orbit using a laser as depicted in FIG. 12 can be accomplished using various apparatuses and techniques, exemplary approaches including one or more of: (a) separating the meibomian gland from the tissue surrounding the orbit by injecting a fluid such as an epinephrine-based fluid therebetween via a needle entry point in a vicinity of the limbus; (h) inserting a fiber optic tip through a treatment located approximately midway along a length of an underlying treatment (e.g., elongated kerf) or treatment grouping (e.g., collection of relatively small treatments approximating, or bounded by, shapes of the illustrated elongated kerfs) and then forming the treatment or treatment grouping in the tissue surrounding the orbit by, for example, changing an orientation of the fiber optic tip as shown in the cross-sectional view of FIG. 12; and (c) inserting a fiber optic tip through a treatment located in a vicinity anywhere between (and/or including) the limbus and a point midway along a length of an underlying treatment or treatment grouping.

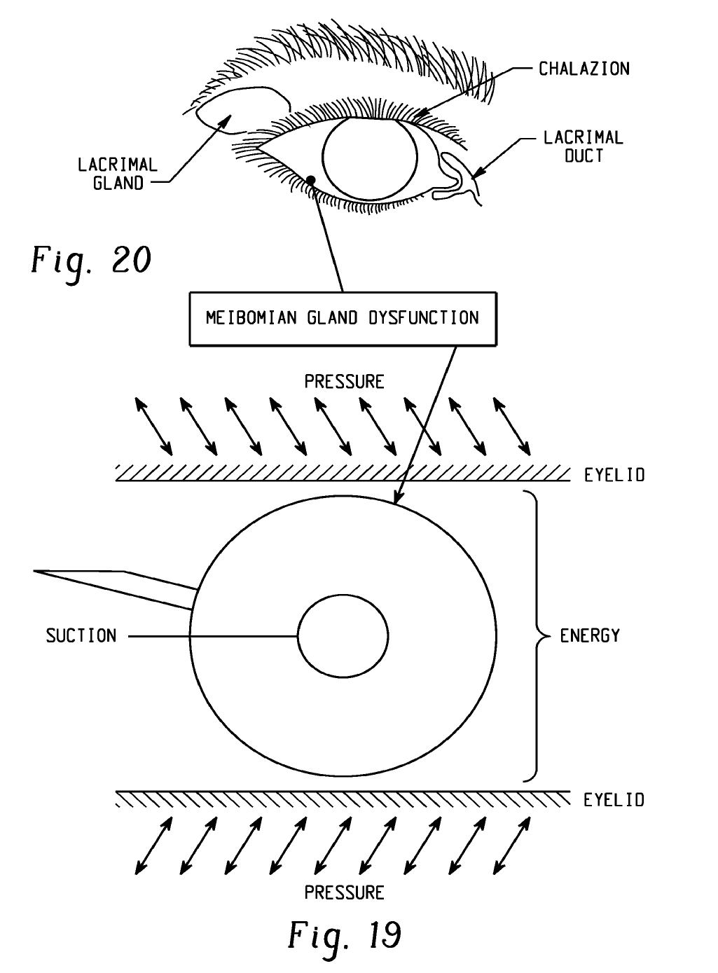

Suction may be applied to the contacting portion, wherein the contacting portion may be constructed and operated as described in connection with FIG. 19. In one illustrative example, movement of the output tip from the center area of the transverse slot in the first direction moves the meibomian gland (e.g., a portion of the meibomian gland) in the first direction and movement of the output tip from the center area of the transverse slot in the second direction move the meibomian gland a portion of the meibomian gland) in the second direction. According to another illustrative example, movement of the output tip from the center area of the transverse slot in the first direction moves a portion of the meibomian gland a corresponding (e.g., approximately equal) distance in the first direction, and movement of the output tip from the center area of the transverse slot in the second direction moves a portion of the meibomian gland a corresponding (e.g., approximately equal) distance in the second direction. Thus, in accordance with an aspect of the present invention, the meibomian gland can be moved (e.g., rotated or torqued) or shifted in two opposing directions to facilitate formation of two different treatments in the underlying tissue surrounding the orbit.

FIG. 19 depicts an embodiment of a meibomian gland displacement device that includes suction. The meibomian gland displacement device may be employed to facilitate, for example, one or more of displacement of the meibomian gland and placement of treatments into the tissue surrounding the orbit. An illustrated embodiment of the meibomian gland displacement device includes a contacting portion and one or more arm implements. According to an exemplary embodiment, the contacting portion can be constructed, for example, to contact a central part of the eye such as the cornea and/or limbus, and the one or more arm implements can be constructed for facilitating positioning on a non-central part of the eye such as over the meibomian gland and tissue surrounding the orbit.

According to modified embodiments, groupings of treatments of the present invention may be disposed around doses (e.g., cuts or kerfs) to the tissue surrounding the orbit implemented in accordance with other technologies. In other modified embodiments, as an alternative or addition to any of the embodiments described herein, treatments may be arranged to approximate or resemble prior-art surgical-formation shapes. For instance, treatments may be applied to resemble, or in combination with, correctional patterns as described in U.S. Pat. No. 6,263,879, the contents of which are expressly incorporated herein by reference. In implementations wherein treatments of the present invention are applied in combination with one or more of the patterns or ablation patterns disclosed in the aforementioned patent, the treatments can be disposed for example along part or all of the boundary(ies) of the linear ablation pattern(s) with or without the ablation pattern(s) being formed as well. In modified embodiments, any of the above treatments may be applied in combination with any other eye treatments to the extent compatible, or modifiable to be compatible, by one skilled in the art, with the present treatments. For instance, the presently-described alterations (e.g., rotations and/or shifts) to the tissue surrounding the orbit in connection with the formation of treatments in the meibomian gland may be modified and/or combined with other technologies (e.g., such as described in the aforementioned patent) involving applications or formations of treatments (e.g., ablations) to the meibomian gland.

According to an implementation of the invention, treating of an eye in need of one or more of a physiological and a vision correction is provided comprising projecting a first form of electromagnetic energy onto tissue surrounding the eye orbit in the form of a spot or a line, and focusing electromagnetic energy through the pattern and into a meibomian gland. The projected pattern can be radial spots, and the electromagnetic energy can be focused onto the tissue surrounding the orbit in the form/shape of the tissue surrounding the orbit radial spots. The projecting in some instances can be preceded by rotating or shifting a portion of the tissue surrounding the orbit, relative to the meibomian gland, from a first configuration to a second configuration, and the focusing can be followed by rotating or shifting at least part of the portion in a direction back to the first configuration.

The method can comprise projecting a second pattern of electromagnetic energy onto a meibomian gland in the form of a second projected radial spot, and focusing electromagnetic energy through the second radial spot and onto the meibomian gland in the form of a second tissue surrounding the orbit radial spot. It may further comprise projecting a third pattern of electromagnetic energy onto a meibomian gland in the form of a third projected radial spot, and focusing electromagnetic energy through the third radial spot and onto the tissue surrounding the orbit in the form of a third radial spot. Performance of the method can incorporate projecting a fourth pattern of electromagnetic energy onto a meibomian gland in the form of a fourth projected radial spot, and focusing electromagnetic energy through the fourth radial spot and onto the tissue surrounding the orbit in the form of a fourth radial spot. Here, the projecting can be preceded by rotating or shifting a portion of the tissue surrounding the orbit, relative to the meibomian gland, from a first configuration to a second configuration, and/or the focusing can be followed by rotating or shifting at least part of the portion in a direction back to the first configuration.

In any of the steps of the preceding paragraphs, the projected pattern can be radial spot, and the electromagnetic energy can be focused onto the meibomian gland in the form of a set of radial spots. The projecting can be preceded here as well by rotating or shifting a portion of the tissue surrounding the orbit, relative to the meibomian gland, from a first configuration to a second configuration, and the focusing can be followed by rotating or shifting at least part of the portion in a direction back to the first configuration. Also, the radial spots can be substantially equally spaced from the projected radial spot, and/or the tissue surrounding the orbit radial spots can be substantially equally spaced from the projected radial spot.

Furthermore, in connection with any of the steps of the preceding paragraphs, performance of the method may comprise projecting a second pattern of electromagnetic energy onto a meibomian gland in the form of a second projected radial line, and focusing electromagnetic energy through the second projected radial line and onto the meibomian gland in the form of a set of second tissue surrounding the orbit radial spots. The performing may further comprise projecting a third pattern of electromagnetic energy onto a meibomian gland in the form of a third projected radial line, and focusing electromagnetic energy through the third projected radial spot and onto the tissue surrounding the orbit in the form of a set of third radial spots. Additional treatment processes may comprise projecting a fourth pattern of electromagnetic energy onto a meibomian gland in the form of a fourth projected radial spot, and focusing electromagnetic energy through the projected fourth radial spot and onto the tissue surrounding the orbit in the form of a set of fourth radial spots. The second, third and fourth radial spots can be substantially equally spaced from the respective second, third and fourth projected radial spots.

Corresponding or related structure and methods disclosed or referenced herein and/or in any and all co-pending, abandoned or patented application(s) naming any of the named inventor(s) or assignee(s) of this disclosure and invention, are incorporated herein by reference in their entireties, wherein such incorporation includes corresponding or related structure (and modifications thereof) which may be, in whole or in part, (i) operable and/or constructed with, (ii) modified by one skilled in the art to be operable and/or constructed with, and/or (iii) implemented/made/used with or in combination with, any part(s) of the present invention according to this disclosure, that of the application and references cited therein, and the knowledge and judgment of one skilled in the art.

Such patents include, but are not limited to U.S. Pat. No. 7,970,030 entitled Dual pulse-width medical laser with presets; U.S. Pat. No. 7,970,027 entitled Electromagnetic energy distributions for electromagnetically induced mechanical cutting; U.S. Pat. No. 7,967,017 entitled Methods for treating eye conditions; U.S. Pat. No. 7,957,440 entitled Dual pulse-width medical laser; U.S. Pat. No. 7,942,667 entitled Electromagnetic radiation emitting toothbrush and dentifrice system; U.S. Pat. No. 7,909,040 entitled Methods for treating eye conditions; U.S. Pat. No. 7,891,363 entitled Methods for treating eye conditions; U.S. Pat. No. 7,878,204 entitled Methods for treating hyperopia and presbyopia via laser tunneling; U.S. Pat. No. 7,867,223 entitled Methods for treating hyperopia and presbyopia via laser tunneling; U.S. Pat. No. 7,817,687 entitled Electromagnetic energy distributions for electromagnetically induced mechanical cutting; U.S. Pat. No. 7,815,630 entitled Target-close electromagnetic energy emitting device; U.S. Pat. No. 7,751,895 entitled Tissue treatment device and method; U.S. Pat. No. 7,702,196 entitled Modified-output fiber optic tips; U.S. Pat. No. 7,697,814 entitled Radiation emitting apparatus with spatially controllable output energy distributions; U.S. Pat. No. 7,696,466 entitled Electromagnetic energy distributions for electromagnetically induced mechanical cutting; U.S. Pat. No. 7,695,469 entitled Electromagnetic energy output system; U.S. Pat. No. 7,665,467 entitled Methods for treating eye conditions; U.S. Pat. No. 7,630,420 entitled Dual pulse-width medical laser; U.S. Pat. No. 7,620,290 entitled Modified-output fiber optic tips; U.S. Pat. No. 7,578,622 entitled Contra-angle rotating handpiece having tactile-feedback tip ferrule; U.S. Pat. No. 7,575,381 entitled Fiber tip detector apparatus and related methods; U.S. Pat. No. 7,561,226 entitled Handpieces having illumination and laser outputs; U.S. Pat. No. 7,467,946 entitled Electromagnetic radiation emitting toothbrush and dentifrice system; U.S. Pat. No. 7,461,982 entitled Contra-angle rotating handpiece having tactile-feedback tip ferrule; U.S. Pat. No. 7,461,658 entitled Methods for treating eye conditions; U.S. Pat. No. 7,458,380 entitled Methods for treating eye conditions; U.S. Pat. No. 7,424,199 entitled Fiber tip fluid output device; U.S. Pat. No. 7,421,186 entitled Modified-output fiber optic tips; U.S. Pat. No. 7,415,050 entitled Electromagnetic energy distributions for electromagnetically induced mechanical cutting; U.S. Pat. No. 7,384,419 entitled Tapered fused waveguide for delivering treatment electromagnetic radiation toward a target surface; U.S. Pat. No. 7,356,208 entitled Fiber detector apparatus and related methods; U.S. Pat. No. 7,320,594 entitled Fluid and laser system; U.S. Pat. No. 7,303,397 entitled Caries detection using timing differentials between excitation and return pulses; U.S. Pat. No. 7,292,759 entitled Contra-angle rotating handpiece having tactile-feedback tip ferrule; U.S. Pat. No. 7,290,940 entitled Fiber tip detector apparatus and related methods; U.S. Pat. No. 7,288,086 entitled High-efficiency, side-pumped diode laser system; U.S. Pat. No. 7,270,657 entitled Radiation emitting apparatus with spatially controllable output energy distributions; U.S. Pat. No. 7,261,558 entitled Electromagnetic radiation emitting toothbrush and dentifrice system; U.S. Pat. No. 7,194,180 entitled Fiber detector apparatus and related methods; U.S. Pat. No. 7,187,822 entitled Fiber tip fluid output device; U.S. Pat. No. 7,144,249 entitled Device for dental care and whitening; U.S. Pat. No. 7,108,693 entitled Electromagnetic energy distributions for electromagnetically induced mechanical cutting; U.S. Pat. No. 7,068,912 entitled Fiber detector apparatus and related methods; U.S. Pat. No. 6,942,658 entitled Radiation emitting apparatus with spatially controllable output energy distributions; U.S. Pat. No. 6,829,427 entitled Fiber detector apparatus and related methods; U.S. Pat. No. 6,821,272 entitled Electromagnetic energy distributions for electromagnetically induced cutting; U.S. Pat. No. 6,744,790 entitled Device for reduction of thermal lensing; U.S. Pat. No. 6,669,685 entitled Tissue remover and method; U.S. Pat. No. 6,616,451 entitled Electromagnetic radiation emitting toothbrush and dentifrice system; U.S. Pat. No. 6,616,447 entitled Device for dental care and whitening; U.S. Pat. No. 6,610,053 entitled Methods of using atomized particles for electromagnetically induced cutting; U.S. Pat. No. 6,567,582 entitled Fiber tip fluid output device; U.S. Pat. No. 6,561,803 entitled Fluid conditioning system; U.S. Pat. No. 6,544,256 entitled Electromagnetically induced cutting with atomized fluid particles for dermatological applications; U.S. Pat. No. 6,533,775 entitled Light-activated hair treatment and removal device; U.S. Pat. No. 6,389,193 entitled Rotating handpiece; U.S. Pat. No. 6,350,123 entitled Fluid conditioning system; U.S. Pat. No. 6,288,499 entitled Electromagnetic energy distributions for electromagnetically induced mechanical cutting; U.S. Pat. No. 6,254,597 entitled Tissue remover and method; U.S. Pat. No. 6,231,567 entitled Material remover and method; U.S. Pat. No. 6,086,367 entitled Dental and medical procedures employing laser radiation; U.S. Pat. No. 5,968,037 entitled User programmable combination of atomized particles for electromagnetically induced cutting; U.S. Pat. No. 5,785,521 entitled Fluid conditioning system; and U.S. Pat. No. 5,741,247 entitled Atomized fluid particles for electromagnetically induced cutting.

Also, the above disclosure and referenced items, and that described on the referenced pages, are intended to be operable or modifiable to be operable, in whole or in part, with corresponding or related structure and methods, in whole or in part, described in the following published applications and items referenced therein, which applications are listed as follows: App. Pub. 20110192405 entitled Methods for treating eye conditions; App. Pub. 20110172650 entitled. Methods for treating eye conditions; App. Pub. 20110165535 entitled Handpiece finger switch for actuation of handheld medical instrumentation; App. Pub. 20110151394 entitled Plaque toothtool and dentifrice system; App. Pub. 20110096802 entitled High power radiation source with active-media housing; App. Pub. 20110096549 entitled High power radiation source with active-media housing; App. Pub. 20110129789 entitled Drill and flavored fluid particles combination; App. Pub. 20110082526 entitled Target-close electromagnetic energy emitting device; App. Pub. 20110059417 entitled Fluid and pulsed energy output system; App. Pub. 20110032958 entitled Electromagnetic energy distributions for electromagnetically induced mechanical cutting; App. Pub. 20100233645 Efficient laser and fluid conditioning and cutting system; App. Pub. 20100185188 entitled Electromagnetically induced treatment devices and methods; App. Pub. 20100167228 entitled Electromagnetic radiation emitting toothbrush and dentifrice system; App. Pub. 20100151407 entitled Device having activated textured surfaces for treating oral tissue; App. Pub. 20100151406 entitled Fluid conditioning system; App. Pub. 20100145323 entitled Electromagnetic energy output system; App. Pub. 20100145323 entitled Electromagnetic energy output system; App. Pub. 20100137852 entitled. Non-contact handpiece for laser tissue cutting; App. Pub. 20100100086 entitled Satellite-platformed electromagnetic energy treatment device; App. Pub. 20100125291 entitled Drill and flavored fluid particles combination; App. Pub. 20100086892 entitled Modified-output fiber optic tips; App. Pub. 20100042082 entitled Methods and devices for treating presbyopia; App. Pub. 20090298004 entitled Tunnelling probe; App. Pub. 20090281531 entitled Interventional and therapeutic electromagnetic energy systems; App. Pub. 20090225060 entitled Wrist-mounted laser with animated, page-based graphical user-interface; App. Pub. 20090143775 entitled Medical laser having controlled-temperature and sterilized fluid output; App. Pub. 20090141752 entitled Dual pulse-width medical laser with presets; App. Pub. 20090105707 entitled Drill and flavored fluid particles combination; App. Pub. 20090104580 entitled Fluid and pulsed energy output system; App. Pub. 20090076490 entitled Fiber tip fluid output device; App. Pub. 20090075229 entitled Probes and biofluids for treating and removing deposits from tissue surfaces; App. Pub. 20090067189 entitled Contra-angle rotating handpiece having tactile-feedback tip ferrule; App. Pub. 20090062779 entitled Methods for treating eye conditions with low-level light therapy; App. Pub. 20090056044 entitled Electromagnetic radiation emitting toothbrush and dentifrice system; App. Pub. 20090043364 entitled Electromagnetic energy distributions for electromagnetically induced mechanical cutting; App. Pub. 20090042171 entitled Fluid controllable laser endodontic cleaning and disinfecting system; WO 2010/051579, entitled Surface structure modification; App. Pub. 20090035717 entitled Electromagnetic radiation emitting toothbrush and transparent dentifrice system; App. Pub. 20090031515 entitled Transparent dentifrice for use with electromagnetic radiation emitting toothbrush system; App. Pub. 20090225060 entitled Wrist-mounted laser with animated, page-based graphical user-interface; App. Pub. 20090143775 entitled Medical laser having controlled-temperature and sterilized fluid output; App. Pub. 20090141752 entitled Dual pulse-width medical laser with presets; App. Pub. 20090105707 entitled Drill and flavored fluid particles combination; App. Pub. 20090104580 entitled Fluid and pulsed energy output system; App. Pub. 20090076490 entitled Fiber tip fluid output device; App. Pub. 20090075229 entitled Probes and biofluids for treating and removing deposits from tissue surfaces; App. Pub. 20090067189 entitled Contra-angle rotating handpiece having tactile-feedback tip ferrule; App. Pub. 20090062779 entitled Methods for treating eye conditions with low-level light therapy; App. Pub. 20090056044 entitled. Electromagnetic radiation emitting toothbrush and dentifrice system; App. Pub. 20090043364 entitled Electromagnetic energy distributions for Electromagnetically induced mechanical cutting; App. Pub. 20090042171 entitled Fluid controllable laser endodontic cleaning and disinfecting system; App. Pub. 20090035717 entitled Electromagnetic radiation emitting toothbrush and transparent dentifrice system; App. Pub. 20090031515 entitled Transparent dentifrice for use with electromagnetic radiation emitting toothbrush system; App. Pub. 20080317429 entitled Modified-output fiber optic tips; App. Pub. 20080276192 entitled Method and apparatus for controlling an electromagnetic energy output system; App. Pub. 20080240172 entitled Radiation emitting apparatus with spatially controllable output energy distributions; App. Pub. 20080221558 entitled Multiple fiber-type tissue treatment device and related method; App. Pub. 20080219629 entitled Modified-output fiber optic tips; App. Pub. 20080212624 entitled Dual pulse-width medical laser; App. Pub. 20080203280 entitled Target-close electromagnetic energy emitting device; App. Pub. 20080181278 entitled Electromagnetic energy output system; App. Pub. 20080181261 entitled Electromagnetic energy output system; App. Pub. 20080157690 entitled Electromagnetic energy distributions for electromagnetically induced mechanical cutting; App. Pub. 20080151953 entitled Electromagnet energy distributions for electromagnetically induced mechanical cutting; App. Pub. 20080138764 entitled Fluid and laser system; App. Pub. 20080125677 entitled Methods for treating hyperopia and presbyopia via laser tunneling; App. Pub. 20080125676 entitled Methods for treating hyperopia and presbyopia via laser tunneling; App. Pub. 20080097418 entitled Methods for treating eye conditions; App. Pub. 20080097417 entitled Methods for treating eye conditions; App. Pub. 20080097416 entitled Methods for treating eye conditions; App. Pub. 20080070185 entitled Caries detection using timing differentials between excitation and return pulses; App. Pub. 20080069172 entitled Electromagnetic energy distributions for electromagnetically induced mechanical cutting; App. Pub. 20080065057 entitled High-efficiency, side-pumped diode laser system; App. Pub. 20080065055 entitled Methods for treating eye conditions; App. Pub. 20080065054 entitled Methods for treating hyperopia and presbyopia via laser tunneling; App. Pub. 20080065053 entitled Methods for treating eye conditions; App. Pub. 20080033411 entitled High efficiency electromagnetic laser energy cutting device; App. Pub. 20080033409 entitled Methods for treating eye conditions; App. Pub. 20080033407 entitled Methods for treating eye conditions; App. Pub. 20080025675 entitled Fiber tip detector apparatus and related methods; App. Pub. 20080025672 entitled. Contra-angle rotating handpiece having tactile-feedback tip ferrule; App. Pub. 20080025671 entitled Contra-angle rotating handpiece having tactile-feedback tip ferrule; App. Pub. 20070298369 entitled Electromagnetic radiation emitting toothbrush and dentifrice system; App. Pub. 20070263975 entitled Modified-output fiber optic tips; App. Pub. 20070258693 entitled Fiber detector apparatus and related methods; App. Pub. 20070208404 entitled Tissue treatment device and method; App. Pub. 20070208328 entitled Contra-angel rotating handpiece having tactile-feedback tip ferrule; App. Pub. 20070190482 entitled Fluid conditioning system; App. Pub. 20070184402 entitled Caries detection using real-time imaging and multiple excitation frequencies; App. Pub. 20070128576 entitled Output attachments coded for use with electromagnetic-energy procedural device; App. Pub. 20070104419 entitled Fiber tip fluid output device; App. Pub. 20070060917 entitled High-efficiency, side-pumped diode laser system; App. Pub. 20070059660 entitled Device for dental care and whitening; App. Pub. 20070054236 entitled Device for dental care and whitening; App. Pub. 20070054235 entitled Device for dental care and whitening; App. Pub. 20070054233 entitled Device for dental care and whitening; App. Pub. 20070042315 entitled Visual feedback implements for electromagnetic energy output devices; App. Pub. 20070016176 entitled Laser handpiece architecture and methods; App. Pub. 20070014517 entitled Electromagnetic energy emitting device with increased spot size; App. Pub. 20070014322 entitled Electromagnetic energy distributions for electromagnetically induced mechanical cutting; App. Pub. 20070009856 entitled Device having activated textured surfaces for treating oral tissue; App. Pub. 20070003604 entitled Tissue coverings bearing customized tissue images; App. Pub. 20060281042 entitled Electromagnetic radiation emitting toothbrush and dentifrice system; App. Pub. 20060275016 entitled Contra-angle rotating handpiece having tactile-feedback tip ferrule; App. Pub. 20060241574 entitled Electromagnetic energy distributions for electromagnetically induced disruptive cutting; App. Pub. 20060240381 entitled Fluid conditioning system; App. Pub. 20060210228 entitled Fiber detector apparatus and related methods; App. Pub. 20060204203 entitled Radiation emitting apparatus with spatially controllable output energy distributions; App. Pub. 20060142745 entitled Dual pulse-width medical laser with presets; App. Pub. 20060142744 entitled Identification connector for a medical laser handpiece; App. Pub. 20060142743 entitled Medical laser having controlled-temperature and sterilized fluid output; App. Pub. 20060126680 entitled Dual pulse-width medical laser; App. Pub. 20060099548 entitled Caries detection using timing differentials between excitation and return pulses; App. Pub. 20060083466 entitled. Fiber tip detector apparatus and related methods; App. Pub. 20060043903 entitled Electromagnetic energy distributions for electromagnetically induced mechanical cutting; App. Pub. 20050283143 entitled Tissue remover and method; App. Pub. 20050281887 entitled Fluid conditioning system; App. Pub. 20050281530 entitled Modified-output fiber optic tips; App. Pub. 20050256517 entitled Electromagnetically induced treatment devices and methods; App. Pub. 20050256516 entitled Illumination device and related methods; App. Pub. 20040106082 entitled Device for dental care and whitening; App. Pub. 20040092925 entitled Methods of using atomized particles for electromagnetically induced cutting; App. Pub. 20040091834 entitled Electromagnetic radiation emitting toothbrush and dentifrice system; App. Pub. 20040068256 entitled Tissue remover and method; App. Pub. 20030228094 entitled Fiber tip fluid output device; App. Pub. 20020149324 entitled Electromagnetic energy distributions for electromagnetically induced mechanical cutting; and App. Pub. 20020014855 entitled Electromagnetic energy distributions for electromagnetically induced mechanical cutting.

All of the contents of the preceding applications are incorporated herein by reference in their entireties, Although the disclosure herein refers to certain illustrated embodiments, it is to be understood that these embodiments have been presented by way of example rather than limitation. For example, any of the radiation outputs (e.g., lasers), any of the fluid outputs (e.g., water outputs), and any conditioning agents, particles, agents, etc., and particulars or features thereof, or other features, including method steps and techniques, may be used with any other structure(s) and process described or referenced herein, in whole or in part, in any combination or permutation as a non-equivalent, separate, non-interchangeable aspect of this invention. Corresponding or related structure and methods specifically contemplated, disclosed and claimed herein as part of this invention, to the extent not mutually inconsistent as will be apparent from the context, this specification, and the knowledge of one skilled in the art, including, modifications thereto, which may he, in whole or in part, (i) operable and/or constructed with, (ii) modified by one skilled in the art to be operable and/or constructed with, and/or (iii) implemented/made/used with or in combination with, any parts of the present invention according to this disclosure, include: (I) any one or more parts of the above disclosed or referenced structure and methods and/or (II) subject matter of any one or more of the following claims and parts thereof, in any permutation and/or combination. The intent accompanying this disclosure is to have such embodiments construed in conjunction with the knowledge of one skilled in the art to cover all modifications, variations, combinations, permutations, omissions, substitutions, alternatives, and equivalents of the embodiments, to the extent not mutually exclusive, as may fall within the spirit and scope of the invention as limited only by the appended claims.

1-20. (canceled)

21. A method for treating at least one meibomian gland, comprising:
a first step of applying electromagnetic energy to the meibomian gland, the electromagnetic energy comprising wavelengths within a range of about 1700 to 2000 microns for absorption by meibum in the meibomian gland, wherein the energy application causes liquefaction of at least a majority of meibum in the meibomian gland without the need for any physical manipulation of the meibomian gland;
applying electromagnetic energy comprising mid-infrared energy onto obstructing materials and/or tissues of the meibomian gland using a radial firing tip; and
applying electromagnetic energy comprising near-infrared energy onto at least a portion of the meibomian gland to stimulate at least a portion of the meibomian gland.

22. The method of claim 21, wherein the electromagnetic energy can be used to remove the outer layer of the meibomian gland.

23. The method of claim 21, wherein the electromagnetic energy can be used to kill bacteria that causes the meibomian gland to become clogged.

24. The method of claim 21, wherein the stimulation comprises using CW energy gated electromagnetic energy.

25. The method of claim 24, wherein the stimulation comprises using continuous wave energy gated on for about 200 ms and off for about 200 ms.

26. The method of claim 21, wherein the stimulation restores the meibomian gland production of meibomia to a relatively more youthful stage.

27. The method of claim 21, wherein the stimulation is also applied to orbital surrounding tissues.

28. The method of claim 21, wherein the radial firing tip comprises a diameter of 100 to 600 microns.
